(19) United States
(12) Patent Application Publication
(10) Pub. No.: US 2022/0273963 A1
(43) Pub. Date: Sep. 1, 2022

(54) Systems and Methods for Aesthetic Treatment

(71) Applicant: Dominion Aesthetic Technologies, Inc., San Antonio, TX (US)

(72) Inventors: John G. Daly, Sorrento, FL (US); Robert E. McKinney, Winter Park, FL (US); Scott R. Marable, Winter Springs, FL (US); Matthew D. Hawk, Altamonte Springs, FL (US); Edward Adamkiewicz, Longwood, FL (US); Eric B. Bagwell, Winter Springs, FL (US)

(73) Assignee: Dominion Aesthetic Technologies, Inc., San Antonio, TX (US)

(21) Appl. No.: 17/744,416

(22) Filed: May 13, 2022

Related U.S. Application Data
(63) Continuation-in-part of application No. 17/017,179, filed on Sep. 10, 2020.
Application No. 17/017,179 is a continuation of application No. 15/820,737, filed on Nov. 22, 2017, now Pat. No. 10,994,151.
Application No. 15/820,737 is a continuation of application No. 16/900,388, filed on Jun. 12, 2020.
(60) Provisional application No. 62/601,674, filed on Mar. 28, 2017.
(60) Provisional application No. 62/497,535, filed on Nov. 22, 2016.
(60) Provisional application No. 62/497,534, filed on Nov. 22, 2016.
(60) Provisional application No. 62/497,520, filed on Nov. 22, 2016.
(60) Provisional application No. 62/497,503, filed on Nov. 22, 2016.
(60) Provisional application No. 62/861,293, filed on Jun. 13, 2019.

(51) Int. Cl. A61N 5/06; A61B 18/20; A61B 18/22

(52) U.S. Cl. CPC A61N 5/0616; A61B 18/203; A61B 18/22; G02B 3/08

(57) ABSTRACT

Provided herein is a multifunctional aesthetic system including a housing, an electromagnetic array situated in the housing and having one or more electromagnetic radiation (EMR) sources, a controller in electronic communication with the array to operate the one or more of the EMR sources to direct the EMR beam to a treatment area, and one or more sensors in electronic communication with the controller for providing feedback to the controller based on defined parameters to allow the controller to adjust at least one operating condition of the multifunctional system in response to the feedback.

### RELATED APPLICATIONS

This application is a continuation-in-part of U.S. application Ser. No. 17/017,179, filed Sep. 10, 2020, which is a continuation of U.S. application Ser. No. 15/820,737, filed Nov. 22, 2017, now U.S. Pat. No. 10,994,151, which claims the benefit of and priority to U.S. Provisional Application No. 62/601,674, filed Mar. 28, 2017, U.S. Provisional Application No. 62/497,535, filed Nov. 22, 2016, U.S. Provisional Application No. 62/497,534, filed Nov. 22, 2016, U.S. Provisional Application No. 62/497,520, filed Nov. 22, 2016, and U.S. Provisional Application No. 62/497,503, filed Nov. 22, 2016, all of which are incorporated herein by reference. This application is a continuation of U.S. application Ser. No. 16/900,388, filed Jun. 12, 2020, which claims the benefit of and priority to U.S. Provisional Application No. 62/861,293, filed Jun. 13, 2019, all of which are incorporated herein by reference.

### TECHNICAL FIELD

The present disclosure relates generally to aesthetic treatment systems, and more particularly, to multifunction aesthetic treatment systems.

### BACKGROUND

Lasers have been applied to medical procedures since they became commercially available in the 1970's. Generally, aesthetic lasers are used for invasive, minimally invasive and non-invasive aesthetic procedures such as, for example, skin treatment and body sculpting. However, with a wide range of wavelengths and power levels, more than 50 different treatment protocols are common. Conventionally, a single laser system is packaged into a single medical device. Thus, conventionally, aesthetic practitioners may require many laser aesthetic treatment systems to perform various procedures. For example, some doctors may require 4, 5, 6, 7, 15, or more laser aesthetic systems to perform procedures requiring different treatment protocols such as, for example, skin ablation/peeling, wrinkle reduction, hyper pigmentation, rosacea, acne, mole removal, skin toning, vein treatments, body sculpting, hair removal, tattoo removal, etc.

Conventional aesthetic laser systems have low efficiency, requiring large power supplies and cooling systems. For example, some conventional laser aesthetic systems incorporate large flash lamp pumped lasers often weighing more than 100 lbs. Diode pumped solid state lasers are more efficient and somewhat smaller, but are expensive and have maintenance issues. Direct Diode lasers offer efficiency and potential low cost, but the need for high amperage power, cooling, and poor beam quality has limited their application.

### SUMMARY

In one embodiment, a multifunctional aesthetic system is provided. The system includes a housing. The system also includes an electromagnetic array situated in the housing and having one or more electromagnetic radiation (EMR) source(s), each EMR source configured to generate an EMR beam having a wavelength different than that of an EMR beam generated by another, if any, of the EMR sources. The system also includes a controller in electronic communication with the array to operate one or more of the EMR sources to direct the EMR beam to a treatment area. The system also includes one or more sensors in electronic communication with the controller for providing feedback to the controller based on defined parameters to allow the controller to adjust at least one operating condition of the multifunctional system in response to the feedback.

In some embodiments, the housing is designed to be portable. In some embodiments, the one or more EMR sources are modularly replaceable within the array to provide customization of, or a combination of wavelengths generated by the one or more EMR sources. In some embodiments, each of the one or more EMR sources is configured to generate an EMR beam having one of an infrared wavelength, a visible light wavelength, or an ultraviolet wavelength. In some embodiments, the controller is configured to operate two or more EMR sources simultaneously, sequentially, or in an alternating pattern to emit the EMR beams from two or more EMR sources. In some embodiments, the controller is configured adjust the at least one operating condition. In some embodiments, the controller is configured to adjust at least one of a flow rate of a cooling airflow impinging on the treatment area, a temperature of the cooling airflow impinging on the treatment area, a spacing between the treatment area and an apparatus directing the cooling airflow onto the treatment area, a power of the EMR beam, a scanning speed of the EMR beam relative to the treatment area, or combinations thereof. In some embodiments, the one or more sensors includes a temperature sensor, the feedback including temperature data indicating a temperature of the skin (or the treatment surface) in the treatment area or the temperature of the skin (or the treatment surface) near the treatment area, wherein the at least one adjusted operating condition is an emitted EMR beam power. In some embodiments, the sensor includes a temperature sensor, the feedback including temperature data indicating a temperature of the treatment area or the temperature near the treatment area, wherein the at least one adjusted operating condition is a flow rate of a cooling airflow directed onto the treatment area. In some embodiments, the sensor includes a temperature sensor, the feedback including temperature data indicating a temperature of the treatment area, wherein the at least one adjusted operating condition is a spacing between the treatment area and an apparatus directing a cooling airflow onto the treatment area. In some embodiments, the sensor is configured to provide the feedback without contacting the treatment area. In some embodiments, the sensor includes a proximity sensor, the feedback including the distance the head is from the treatment area, wherein the adjusted operating condition is the spacing between the treatment area and the head. In some embodiments, the temperature of the skin is used to calculate the temperature of the subcutaneous region by using models which include information such as the heat flux through the skin.

In some embodiments, the system also includes an EMR pathway directing the EMR to the treatment area. In some embodiments, the pathway also includes two or more optically separated output fibers to permit simultaneous or sequential illumination of a target area by two or more different wavelengths. In some embodiments, the system also includes a device optically engaged with the pathway for modifying the EMR beam received from the pathway to direct the EMR beam onto the treatment area. In some embodiments, the device also includes an optical element for expanding the EMR beam to direct the EMR beam onto an expanded treatment area. In some embodiments, the device also includes a Fresnel or similar lens for focusing the expanded beam to prevent or minimize expansion of the EMR beam in a subsurface treatment region below the treatment area. In some embodiments, the device also includes a beam splitter optically engaged between the pathway and the device for generating a plurality of output beams, wherein the plurality of output beams are emitted by the device to impinge on the treatment area separately and to completely, partially, or to not overlap at a predetermined distance below the treatment area to treat a subsurface treatment region. In some embodiments, the device is optically engaged with a plurality of optically separate portions of the EMR pathway for generating a plurality of output beams, wherein the plurality of output beams are emitted by the device to impinge on the treatment area separately or to partially or completely overlap at a predetermined distance below the treatment area to treat a subsurface treatment region. In some embodiments, the array also includes at least two of the EMR sources each configured to generate an EMR beam having a same wavelength for being directed to the device by the optically separate portions of the pathway. In some embodiments, the device is engaged with one or more sensors for providing feedback associated with the treatment area. In some embodiments, the device is configured to direct a cooling airflow onto the treatment area without disrupting the EMR beam. In some embodiments, the device is configured to direct the EMR beam onto the treatment area, direct the cooling airflow onto the treatment area, and provide the sensor feedback associated with the treatment area without contacting the treatment area. In some embodiments, the system also includes an apparatus engaged at a first end with the housing and engaged at a second end with the device to position the device to direct the EMR beam onto the treatment area. In some embodiments, the apparatus also includes an articulable arm to position the device. In some embodiments, the apparatus is configured to receive a signal from the controller to instruct a movement of the apparatus to position the device with respect to the treatment area. In some embodiments, the apparatus is configured to receive the signal from the controller responsive to feedback received at the controller from the one or more sensors, wherein the sensor may include a position sensor, the feedback including position data indicating a position of the device relative to the treatment area, wherein the at least one adjusted operating condition is a position of the device. In some embodiments, the system also includes a chiller for chilling at least one of the EMR sources or a cooling airflow during operation. In some embodiments, the system also includes a second chiller for chilling another of the at least one of the EMR sources or the cooling airflow during operation.

In another embodiment, a method for aesthetic treatment using a multifunctional system is provided. The method includes operating, by a controller in electronic communication with an electromagnetic array situated in a housing, two or more electromagnetic radiation (EMR) sources of the array to direct an EMR beam generated by each EMR source to a treatment area, each EMR source configured to generate an EMR beam having a wavelength different than that of an EMR beam generated by another of the EMR sources. The method also includes providing, by one or more sensors in electronic communication with the controller, feedback to the controller based on defined parameters. The method may also include adjusting, by the controller, at least one operating condition of the multifunctional system in response to the feedback.

In some embodiments, each EMR source is configured to generate an EMR beam having one of an infrared wavelength, a visible light wavelength, or an ultraviolet wavelength. In some embodiments, the step of operating further comprises operating the two or more EMR sources simultaneously, sequentially, or in an alternating pattern to emit the EMR beams from the two or more EMR sources. In some embodiments, the step of adjusting further comprises maintaining the treatment area at a therapeutically acceptable temperature. In some embodiments, maintaining the treatment area at a therapeutically acceptable temperature includes adjusting at least one of a flow rate of a cooling airflow impinging on the treatment area, a temperature of the cooling airflow impinging on the treatment area, a spacing between the treatment area and a cooling apparatus directing the cooling airflow onto the treatment area, a power of the EMR beam, a scanning speed of the EMR beam relative to the treatment area, or combinations thereof.

In some embodiments, the method also includes directing the EMR beam along an EMR pathway onto the treatment area. In some embodiments, the method also includes modifying the EMR beam in a device optically engaged with the pathway to direct the EMR beam onto the treatment area. In some embodiments, the step of modifying also includes expanding, by an optical element of the device, the EMR beam to direct the EMR beam onto an expanded treatment area. In some embodiments, the step of modifying also includes focusing, by a Fresnel or similar lens, the expanded beam to prevent or minimize expansion of the EMR beam in a subsurface treatment region below the treatment area. In some embodiments, the step of modifying also includes splitting, by a beam splitter optically engaged between the pathway and the device, the EMR beam to generate a plurality of output beams. In some embodiments, the step of modifying also includes emitting, by the device, the plurality of output beams to impinge on the treatment area separately and to overlap at a predetermined distance below the treatment area to treat a subsurface treatment region. In some embodiments, the step of modifying also includes optically engaging the device with a plurality of optically separate portions of the EMR pathway to generate a plurality of output beams. In some embodiments, the step of modifying also includes emitting, by the device, the plurality of output beams to impinge on the treatment area separately and to overlap at a predetermined distance below the treatment area to treat a subsurface treatment region.

In some embodiments, the method also includes directing, to the device by the optically separate portions of the pathway, at least two EMR beams having a same wavelength, wherein the array includes at least two EMR sources each configured to generate EMR beams having a same wavelength. In some embodiments, the method also includes directing, via the device, a cooling airflow onto the treatment area without disrupting the EMR beam. In some embodiments, the steps of directing, by the device, the EMR beam onto the treatment area, directing, via the device, the cooling airflow onto the treatment area, and providing, by the one or more sensors, feedback to the controller are performed without contacting the device or the sensor with the treatment area. In some embodiments, the step of adjusting also includes controlling, by the controller, a movement of an apparatus engaged with the housing to position the EMR beam with respect to the treatment area. In some embodiments, the step of adjusting also includes moving the apparatus in response to the feedback to reposition EMR beam.

In accordance with example embodiments of the present invention, a method for providing an aesthetic treatment is provided. The method includes providing a plurality of markings to identify boundaries of a treatment area, registering, by an aesthetic treatment device, the plurality of markings to map the treatment area, the aesthetic treatment device having a source for directing an electromagnetic radiation (EMR) beam, and activating the source to generate the EMR beam at the mapped treatment area.

In accordance with aspects of the present invention, the aesthetic treatment device further includes a housing, a treatment arm, with two ends, connected to the housing at one end, a treatment head connected to the treatment arm at the other end, a controller, a system for directing the EMR beam to the treatment head, and a user interface for allowing a user to input data. The treatment head may not contact a surface of the treatment area during delivery of the EMR. The treatment head can include a lens that converts the EMR beam into a rectangular shape with a length and a width. The aesthetic treatment device can further include a system for providing air to the treatment head for delivery to the treatment area. The treatment zone can have a length and a width, with the length being approximately a whole number multiple of the length of the EMR beam and the width being approximately a whole number multiple of the width of the EMR beam.

In accordance with aspects of the present invention, the treatment area is a rectangular shape. The treatment area is set by moving the treatment head to a first corner of the treatment area and registering the first corner, moving the treatment head to a second corner of the treatment area and registering the second corner, moving the treatment head to a third corner of the treatment area and registering the third corner, and moving the treatment head to a fourth corner of the treatment area and registering the fourth corner. The method can further include aligning an alignment light of the aesthetic treatment device with one of the plurality of markings to initiate the registering of the treatment area. The method can further include moving the treatment head in response to an input into at least one of a joystick and the user interface.

In accordance with example embodiments of the present invention, a multifunctional aesthetic system for causing thermal apoptosis in subcutaneous fatty tissues is provided. The system includes an electromagnetic radiation (EMR) source to generate an energy beam and an energy delivery device for directing the energy beam over a first treatment zone in a treatment area while moving the electromagnetic radiation (EMR) source within the treatment zone at a rate that allows subcutaneous tissue to reach a target temperature range. The energy delivery device continues the application of the energy beam to the first treatment zone while keeping the subcutaneous tissue within the target temperature range and the energy delivery device discontinues the application of the energy beam to any of the subcutaneous tissue in the first treatment zone that have been in the target temperature range for a target treatment period of time.

In accordance with aspects of the present invention, the target temperature range of the of the subcutaneous tissue is 42° C.-51° C. The application of the energy beam can be applied to an area that is smaller than the area of the first treatment zone. The energy delivery device can apply less of energy after the subcutaneous tissue has reached the target temperature range than the application of the energy applied prior to the subcutaneous tissue reaching the target temperature range. The application of energy to the first treatment zone can be stopped when the temperature of the treatment zone surface is higher than a maximum surface temperature and the application of energy to the first treatment zone can be restarted when the temperature of the treatment zone surface is lower than the maximum surface temperature. The energy delivery device can apply the application of the energy beam and cooling air to the first treatment area while moving the electromagnetic radiation (EMR) source within the first treatment area to raise a temperature of the subcutaneous tissue to the target temperature range and the energy delivery device can stop the application of the energy beam to the first treatment area while maintaining the cooling air while moving the electromagnetic radiation (EMR) source within the first treatment area.

In accordance with example embodiments of the present invention, an aesthetic apparatus is provided. The aesthetic apparatus device includes an electromagnetic radiation (EMR) source configured to generate an EMR beam, a device for directing the EMR beam and an airflow to a treatment area, a lens for collimating the EMR beam, and a refractive diffuser for transforming the collimated EMR beam into a square EMR beam and produce a uniform energy distribute for uniform tissue heating.

In accordance with aspects of the present invention, the apparatus further includes an air system having a source of air and a cooling system for directing a volume of air at a target velocity sufficiently enough to provide impingement cooling on a tissue surface from the source of air to a treatment area. The apparatus can further include a sensor array having at least one of a skin temperature sensor, an air-cooling temperature sensor, air flow sensor, laser power sensor, a location sensor, and a proximity sensor. The energy delivery device can further include a blocking filter to filter light that reaches the proximity sensor increase accuracy of laser detection for proximity of the apparatus to a surface of the skin.

### BRIEF DESCRIPTION OF THE DRAWINGS

Illustrative, non-limiting example embodiments will be more clearly understood from the following detailed description taken in conjunction with the accompanying drawings.

FIG. 1 is a block diagram illustrating a multifunction system in accordance with an embodiment of the present invention.

FIG. 2 is a perspective view of electromagnetic radiation emission components of a multifunction system in accordance with an embodiment of the present invention.

FIG. 3 is an interior view of a beam combiner of a multifunction system in accordance with an embodiment of the present invention.

FIG. 4 is a schematic view of power and control electronics of a multifunction system including a plurality of EMR drivers in accordance with an embodiment of the present invention.

FIG. 5 is a perspective view of a cooling system of a multifunction system in accordance with an embodiment of the present invention.

FIG. 6 is a perspective view of a cooling mount of a multifunction system in accordance with an embodiment of the present invention.

FIG. 7 is a perspective view of a refrigeration unit of a cooling system of a multifunction system in accordance with an embodiment of the present invention.

FIG. 8 is a perspective view of a two degree of freedom positioning apparatus in accordance with an embodiment of the present invention.

FIG. 9 is a perspective view of a six degree of freedom positioning apparatus in accordance with an embodiment of the present invention.

FIG. 10 is a schematic view of a subcutaneous temperature prediction system in accordance with an embodiment of the present invention.

FIG. 11 is a human tissue profile showing expected penetration depth of various EMR wavelengths in accordance with an embodiment of the present invention.

FIG. 12 is a schematic view of a multifunction system including a switching device in accordance with an embodiment of the present invention.

FIG. 13 is a schematic view of a FET circuit of a switching device in accordance with an embodiment of the present invention.

FIG. 14A is a perspective view of a fiber combiner for providing two separate output paths in accordance with an embodiment of the present invention.

FIG. 14B is a detail view of the fiber combiner of FIG. 14A in accordance with an embodiment of the present invention.

FIG. 15 is a cross-sectional view of a device having split, angled EMR beam delivery in accordance with an embodiment of the present invention.

FIG. 16A is a cross-sectional view of a device having beam shaping optics in accordance with an embodiment of the present invention.

FIG. 16B is a cross-sectional view of the device of FIG. 16A having an adjustable optical element in accordance with an embodiment of the present invention.

FIG. 16C is a cross-sectional view of the device of FIG. 16A having an additional optical element in accordance with an embodiment of the present invention.

FIG. 17 is a perspective view of a device having non-contact sensors in accordance with an embodiment of the present invention.

FIG. 18 is a diagram of a treatment region in accordance with an embodiment of the present invention.

FIG. 19 is a block diagram of an aesthetic treatment method in accordance with an embodiment of the present invention.

FIG. 20 is an embodiment of the treatment system in accordance with an embodiment of the present invention.

FIG. 21 is an exploded view of some components of the treatment device in accordance with an embodiment of the present invention.

FIG. 22 is a view of some components of the treatment device in accordance with an embodiment of the present invention.

FIG. 23 is a schematic of an embodiment of the air chilling system in accordance with an embodiment of the present invention.

FIG. 24 is a schematic of an embodiment of the laser cooling system in accordance with an embodiment of the present invention.

FIG. 25 is a view of the treatment device in accordance with an embodiment of the present invention.

FIGS. 26A, 26B, and 26C show various templates used to mark a treatment area in accordance with an embodiment of the present invention.

### DETAILED DESCRIPTION

Various exemplary embodiments will be described more fully hereinafter with reference to the accompanying drawings, in which some example embodiments are shown. The present disclosure may, however, be embodied in many different forms and should not be construed as limited to the example embodiments set forth herein. Rather, these example embodiments are provided so that this disclosure will be thorough and complete, and will fully convey the scope of the present disclosure to those skilled in the art. In the drawings, the sizes and relative sizes of layers and regions may be exaggerated for clarity. Like numerals refer to like elements throughout.

Unless otherwise defined, all terms, including technical and scientific terms, used herein have the same meaning as commonly understood by one of ordinary skill in the art to which this disclosure belongs. For example, when an element is referred to as being “operatively engaged” with another element, the two elements are engaged in a manner that allows electrical and/or optical communication from one to the other.

Embodiments of the present disclosure generally provide multifunction aesthetic systems. In particular, in some embodiments, the systems of the present disclosure can include one or more electromagnetic radiation (EMR) sources and optionally a beam combiner for combining electromagnetic radiation beams emitted by two sources. In this manner, in some embodiments, the multifunction aesthetic system can emit multiple wavelengths of electromagnetic radiation through a single output device. In some embodiments, the multiple wavelengths can be emitted simultaneously, in alternating pulses, and/or sequentially to permit multiple treatments to be performed by the same multifunction aesthetic system. In some embodiments, the multiple treatments can be performed sequentially, simultaneously, or in alternating fashion.

As used herein, EMR can refer to electromagnetic radiation having any desired wavelength. In particular, EMR generated and/or emitted by embodiments of the present disclosure can be any suitable wavelength, including, for example, visible light, ultraviolet radiation, x-ray radiation, infrared radiation, microwave radiation, radio waves, or combinations thereof.

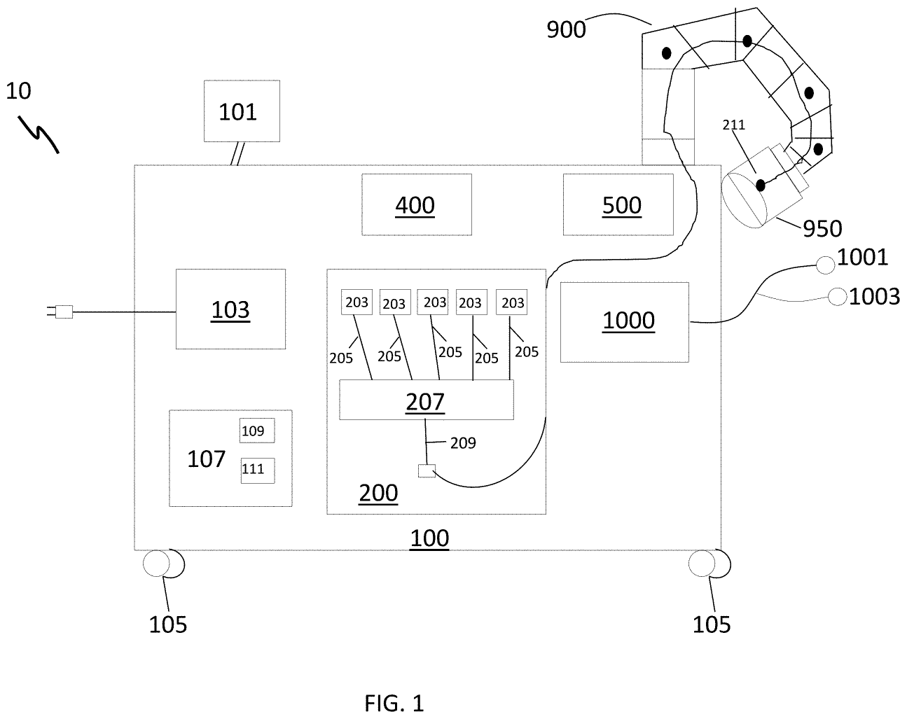

Referring now to FIG. 1, a multifunction aesthetic system 10 can be provided for performing a variety of aesthetic procedures in a single medical device. The system 10 can include a housing 100 for housing, retaining, mounting, or engaging components of the system 10. In some embodiments, the housing 10 can be constructed of any suitable material for providing structural support to and protection of components housed, retained, mounted, or engaged in, on, or with the housing 100, including, for example, plastics, polymers, metals, or any other medically compliant material. To the extent that it is desired to move the system 10, for example, from one exam room or operating room to another, the housing 100 can include one or more wheels 105 to provide mobility of the system 10. To the extent that power is required to be delivered to the system 10, the housing 100 can include one or more power cords 103 for engagement with an AC power source such as, for example, a wall outlet.

In some embodiments, the system 10 can include a user interface 101 electronically connected to the housing 100 for receiving a user input. The user interface 101 can include, for example, an electronic display, a touch-screen monitor, a keyboard, a mouse, any other device or devices capable of receiving input from a user, or combinations thereof. The user input can include, for example, patient data such as height, weight, skin type, age, etc. as well as procedural parameters such as desired beam power, procedure type, wavelength or wavelengths to be applied, pulse duration, treatment duration, beam pattern, treatment area temperature limit, etc.

In some embodiments, the system 10 can also include a computing device 107 for receiving and storing the user input from the user interface 101, for storing and executing appropriate procedure protocols according to the user input, for providing control instruction to various components of the system 10 and receiving feedback from the various components of the system 10. The computing device 107 can be any suitable computing device such as, for example, a laptop, a desktop, a server, a smartphone, a tablet, a personal data assistant, or any other suitable computing device having a memory 109 and a processor 111. The memory 109, in some embodiments, can be any suitable memory 109 for storing electronic data, including the user input data and operational data associated with one or more components of the system 10. The memory 109 can include, for example, random access memory (RAM), flash memory, solid state memory, a hard disk, a non-transitory computer readable medium, any other form of electronic memory, or combinations thereof. The processor 111, in some embodiments, can be any processor suitable for receiving user input from the user interface 101, generating commands for operation of one or more system 10 components, executing any software stored in the memory 109, or combinations thereof. The processor, in some embodiments, can include one or more of a microprocessors, an integrated circuit, an application specific integrated circuit, a microcontroller, a field programmable gate array, any other suitable processing device, or combinations thereof.

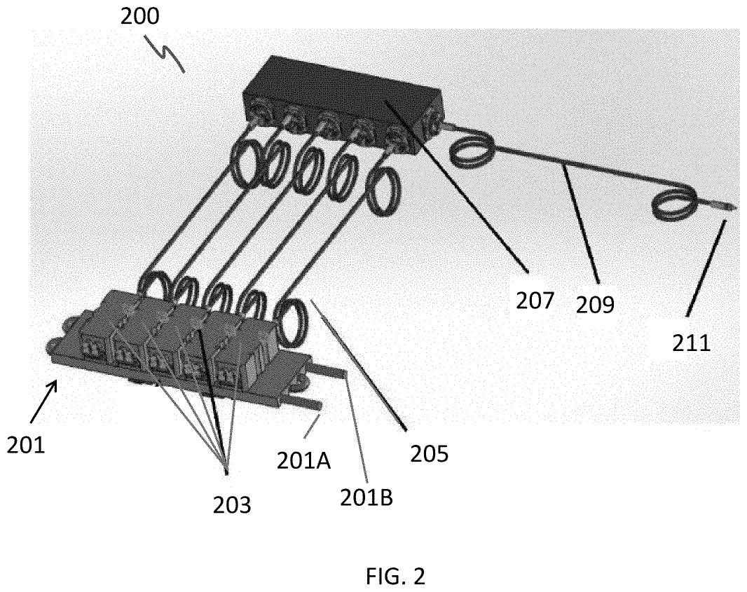

As shown in FIG. 1, the system 10 can also include an electromagnetic array 200. Referring now to FIG. 2, the electromagnetic array 200 can include a mount 201 for mounting a plurality of electromagnetic radiation (EMR) sources thereon. For example, as shown in FIG. 2, the mount 201 includes one or more laser sources 203 mounted thereon. The mount 201, in some embodiments, can include any plate, housing, bracket, or other structure for mounting one or more laser sources 203 thereto. As shown in FIG. 2, in some embodiments, the mount 201 can be a cold plate for providing cooling to the laser sources 203 mounted thereto. For example, as illustrated by FIG. 2, the mount 201 can provide first and second coolant ports 201a, 201b for permitting circulation of a coolant through the mount 201. The coolant can then chill the mount 201, thereby providing a heat sink for cooling the one or more laser sources 203 mounted to the mount 201.

In some embodiments, each laser source 203 can be configured to emit EMR at a particular wavelength. For example, in some embodiments, each laser source 203 can emit EMR at a wavelength between about 200 nm to about 4500 nm. However, it will be apparent in view of this disclosure that each laser source 203 can emit EMR at any desired wavelength in accordance with various embodiments. Furthermore, it will be apparent in view of this disclosure that, in addition to laser sources 203, any other source of electromagnetic radiation having any wavelength can be used in accordance with various embodiments. For example, in some embodiments, EMR sources of the system 200 can emit electromagnetic radiation having any suitable wavelength, including, for example, visible light, ultraviolet radiation, x-ray radiation, infrared radiation, microwave radiation, or radio waves. Thus, because each laser source 203 can be configured to emit a different particular wavelength, just one system 10 can produce EMR beams at wavelengths or combinations of wavelengths required for any one of a plurality of procedures having disparate treatment protocol requirements. Accordingly, in some embodiments, the system can include laser sources 203 emitting wavelengths suitable for performing one or more procedures including, for example, but not limited to, fat reduction, body skin tightening, facial skin tightening, skin resurfacing, skin remodeling, vein reduction or removal, facial pigment removal or reduction, hair removal, acne treatment, scar reduction and removal, psoriasis treatment, stretch mark removal, nail fungus treatment, leukoderma treatment, tattoo removal, or combinations thereof.

Some aesthetic procedures may only require a single wavelength. For example, for some fat reduction procedures, a laser source 203 can be provided which is capable of emitting EMR at a wavelength of about 1064 nm (e.g., about 400 nm to about 3000 nm or about 900 nm to about 1100 nm) can be selected for hyperthermia or apoptosis of fat tissue because it exhibits good transmission through the skin, epidermis, and dermis and deposits energy within the fat cells. On the other hand, skin tightening generally requires other wavelengths that exhibit higher absorption in the epidermis and dermis, where the collagen resides. Thus, for example, a wavelength of about 1320 nm (e.g., about 400 nm to about 3000 nm or about 1300 nm to about 1500 nm) can be used for some body skin tightening procedures. These EMR beam wavelengths deposit more energy to the collagen, creating apoptosis or necrosis and eventually skin tightening from new collagen regrowth.

In other examples, such as for some facial pigment reduction or removal procedures and some vein reduction or removal procedures, for example, a laser source capable of emitting EMR at about 532 nm (e.g., about 500 nm to about 650 nm) can be provided.

Additionally, some aesthetic procedures or combinations of procedures may require two or more wavelengths. For example, to combine the fat reduction and body skin tightening procedures discussed above, a first laser source 203 capable of emitting EMR at 1064 nm and a second laser source 203 capable of emitting EMR at 1320 nm can be provided. In another example, for some facial skin tightening procedures, for example, a first laser source 203 capable of emitting EMR at about 1320 nm (e.g., 400 nm to about 3000 nm or about 1300 nm to about 1500 nm) and a second laser source 203 capable of emitting EMR at about 1470 nm (e.g., 400 nm to about 3000 nm or about 1300 nm to about 1500 nm) can be provided.

To provide additional functionality and facilitate ease of maintenance, in some embodiments, the one or more laser sources 203 can be removably mounted to the mount 201 to permit modular replacement of the laser sources 203. Thus, in such modular configurations, individual laser sources 203 can be replaced, for example, to provide additional or different wavelengths or wavelength combinations as needed for particular procedures. However, it will be apparent in view of this disclosure that, in some embodiments, the one or more laser sources 203 can be permanently attached to the mount 201.

The one or more laser sources 203, in some embodiments, can include one or more fiber coupled lasers. For example, in accordance with various embodiments, the laser sources 203 can include one or more fiber coupled diode lasers and/or flashlamp or diode pumped lasers such as Er:YAG, Er,Cr:YSGG, Nd:YAG, Nd:glass; Er:glass, or any other suitable fiber coupled EMR source. In some embodiments, fiber coupled laser sources 203 can be rated as continuous wave (CW) devices operating at 50 W, 100 W, etc. Such CW devices can be operated in a gated mode where the pulse energy is equal to the pulse duration times the power. Therefore, a 100 W diode laser gated to operate for 5 milliseconds will have pulse energy of 500 mJ. In cases where more pulse energy is required but, for example, power supply or cooling capacity limits the average power, fiber coupled laser sources 203 can be configured as a quasi-CW device. Such quasi-CW devices can produce higher power pulses for the same average power draw by operating at a lower pulse frequency rate. In some embodiments, a quasi-CW device can produce pulses having up to 10 times the average power draw. Thus, for example, a 1000 W/100 W quasi-CW diode would be capable of pulsed operation at 5 milliseconds with 5 Joules per pulse, but limited to one tenth the pulse frequency of a CW laser.

In some embodiments, at least one of the laser sources 203 can include a fiber coupled diode laser. Such laser systems can advantageously operate at efficiencies exceeding 50%, are relatively small in size, draw relatively low power, and exhibit wide wavelength diversity. Fiber coupled diode lasers can, for example, be driven by less than 2.0 volts DC to produce an output of 10 kW or more. Furthermore, such laser sources 203 can be small and lightweight, with the module weighing about 500 grams per 1 kW. In one embodiment, at least one of the laser sources 203 can be a 75 W fiber coupled diode having a size of about 8×4×3 cm (less than 100 cm³). In some embodiments, such laser sources 203 can be used to perform an aesthetic procedure while drawing less than 100 Watts of power. Such low power draw can, in some embodiments, reduce the amount of cooling required, permitting smaller, quieter, more efficient cooling systems.

The compliance voltage for nearly all diodes of interest is slightly less than 2.0 VDC. Packaging and differing bias voltage configurations can be applied to result in a common higher voltage which then allows a lower drive current. For example, a typical 50 W diode driven at 2.0 VDC can require a minimum threshold current of 8 amps to 12 amps and can require more than 60 to 70 amps to produce a desired power level. Such high current necessitates heavy gauge wiring such as #6- or #8-gauge wires to avoid voltage drop, preserve system reliability, and minimize Joule heating. To reduce the required current supply and wiring size, in some embodiments, the diode of each fiber coupled diode laser source 203 can be configured to operate with a common compliance voltage such as, for example, 20 VDC or 25 VDC, with a drive current controlled to match the laser selected and the required output power. By increasing the common compliance voltage to 20 or 25 VDC, the maximum drive current required to operate each laser source 203 can be limited to about 10 amps or less for most aesthetic procedures. By reducing required current, smaller gauge wiring can be used to improve reliability. In some embodiments, such an approach permits use of a single power supply to drive the one or the more than one laser sources 203 by manifolding the power supply into connections with the one or more than one EMR sources. Thus, for example, in embodiments where only one laser is operated at a time, then the system 10 may be provided with only one power supply.

Typical diode packaging employs semiconductor bars with compliance voltages near 2.0 VDC, where threshold currents are in the 8 to 12 amperage range. To reach significant power levels, such diodes can operate as high as 70 amps. The associated problem with these voltage drops and joule heating (I²*R) adds to reliability concerns. However, partial diode bars (i.e., diode bars having a shorter length than a standard 2.0 VDC diode bar) typically require less current proportional to the bar fraction. Thus, by using partial diode bars connected in series, delivering lower current but at a higher voltage for activating each of the partial diodes, required current can be reduced while power is maintained.

In some embodiments, at least one of the laser sources 203 can include a flashlamp or diode pumped laser. For example, many aesthetic skin treatments require application of EMR having a wavelength near 3000 nm, such as, for example, wavelengths greater than 2500 nm. Such wavelengths are typically produced by flashlamp or diode pumped solid state laser devices such as Er:YAG, which produces EMR having a wavelength of about 2940 nm or Er:YSGG, which produces EMR having a wavelength of about 2790 nm. However, although shown and described herein with reference to fiber coupled diode lasers and flashlamp or diode pumped lasers, it will be apparent in view of this disclosure that any suitable type of EMR source capable of being coupled to a fiber optic output cable can be used in accordance with various embodiments. In some embodiments, laser sources 203 including the flashlamp or diode pumped solid state laser devices can also be configured to operate at the common compliance voltage as explained above with reference to the fiber coupled diode lasers. Thus, the system 10, in some embodiments, can use the common power source as discussed above with reference to the fiber coupled diode lasers.

Still referring to FIG. 2 the electromagnetic array 200 can also include a fiber optic relay cable 205 coupled to each of the one or more laser sources 203 for transmitting or relaying the EMR (also referred to as “EMR energy” or “beam”) emitted by the respective laser source 203. The present disclosure refers to EMR, energy, beam, or laser interchangeable throughout the description. In general, each fiber optic relay cable 205 can be constructed of any fiber optic material capable of transmitting EMR having a wavelength emitted by each respective laser source 203. In some embodiments, each fiber optic relay cable 205 can be constructed of, for example, low-OH silica fiber core cables, which transmit wavelengths in a range of about 200 nm to about 2400 nm, Zirconium Fluoride (ZrF4) and/or high purity Chalcogenide glass cables, which transmit wavelengths in a range of about 285 nm to about 4500 nm, or sapphire cables, which transmit wavelengths in a range of about 170 nm to about 5500 nm.

In some embodiments, the fiber optic relay cables 205 can be mated to the laser sources 203 by a fiber optic connector such as, for example, a SMA 905 connector or any other suitable connector. For each of the fiber optic relay cables, the fiber core diameter can be driven by the coupling efficiency of the diode driver and the required power. For example, in CW operation, in one embodiment, for near infrared wavelength ranges, the core diameter can be determined by an energy density limit in the cable of about 1.4 MW/cm² to provide a reliable relay. This reliability limit on the fiber predicts that a 100-micron core diameter can handle up to 85 W and a 400 micron core diameter can be used up to 1300 W. Shorter wavelengths typically scale to lower power limits. Additionally, for pulsed operation where the pulse duration is less than one (1) microsecond (1×10⁻⁶ seconds), fiber damage is not thermal but caused by dielectric breakdown and occurs at lower levels proportional to the pulse duration. That is, although average power is low enough to prevent overheating of the fiber, the power delivered during a pulse duration of less than one (1) microsecond can cause breakdown of the dielectric materials of the fiber. More generally, by selecting the proper fiber core diameter and connectors capable of handling maximum expected power loadings, safe and reliable routing of the EMR power generated by the laser sources 203 is possible.

Still referring to FIG. 2, the system may also include a beam combiner 207 for combining the EMR beams produced by each laser source 203 and transmitted by each relay cable 205 into a single output. Generally, the beam combiner 207 can be any device or system capable of combining several EMR beams of different wavelengths into one output. For example, in some embodiments, the beam combiner can include, for example, fiber switching devices, free-space fiber combiners, butt-coupled combiners, tapered fibers, bundled fibers, and fused fibers.

For example, free space combiners can be packaged with mirrors and gratings to fold separate beams into one fiber. Butt-coupled fiber combiners can mate smaller core fibers into a larger core output cable. For butt-coupled fiber combiners, the smaller fibers are stripped to their cladding and packaged as close to each other as possible, for example, in a circular footprint. The polished fiber ends can be mated (butt-coupled) to a larger fiber core with a diameter greater than the multiple fiber footprint. Tapered fibers can be used to reduce the core diameter of the combined fibers. That is, tapered fibers can be stretched such that the diameter of each tapered fiber is reduced to permit a higher packaging density for fiber coupling. Fiber fusing can be used to mate multiple fibers together by stripping the fibers and bundling them into a close-packed cross-section. The fibers can then be heated and melted to fuse into a single output fiber. Bundled fiber cables can also be used to route multiple sources into one output path. Bundled fibers, in general, can be larger diameter fiber cables formed from many small, individual fibers closely packed within the cable. In embodiments where there is only one laser source 203, common output cable 209 may be a continuation or extension of relay cable 205 if there is no need for beam combiner 207.

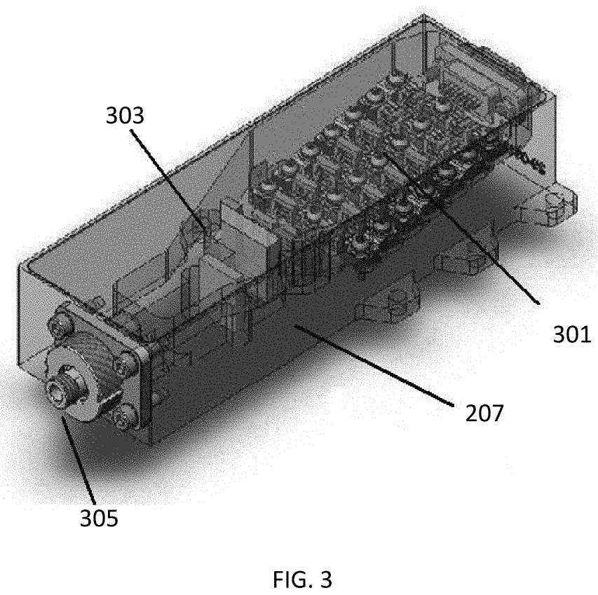

Additionally, as shown in FIG. 3, in some embodiments, the beam combiner 207 can include a high brightness/low cost fiber coupling package such as the device produced for nLight Corporation under NASA SBIR program 05-II S6.02-8619. The device can include multiple diodes 301 all coupled into a single core fiber output port 305. The beam combining optics 303 can be configured to converge each of the individual diode 301 outputs into a common optical path. The beam combiner can then route the converged outputs to an output port 305 (e.g., a SMA 905 connector). The beam combiner 207, in some embodiments, can be configured to combine diverse beam wavelengths for beam powers ranging from a few Watts to more than 10 kW.

In such embodiments, because only the laser sources 203 producing the desired wavelengths are activated at any time, the beam combiner 207 can be a passive device, rather than an active fiber switch. Having a passive device also helps in defining the power limits for the fibers, where the limit in watts for the fibers can be matched to the highest power laser source 203 available where only a single laser source 203 is active at a time, rather than a sum from each laser source 203. To the extent that multiple laser sources 203 are activated simultaneously, the power limit of the combined fibers must be equivalent to at least the sum of the power required to operate each active laser source 203. Alternatively, in some embodiments, the beam combiner 207 can also include one or more fiber switches to selectively output particular wavelengths.

The beam combiner 207 can then output the combined beam to a common output cable 209 coupled to the beam combiner 207 for transmitting or relaying the EMR (also referred to as “treatment energy” or “beam”) combined in the beam combiner 207. Advantageously, the common output cable 209 can permit the different beams produced by the laser sources 203 to be emitted through a single optical device. In particular, by combining or directing the beams in the beam combiner 207 to the common output cable 209, a single optical device of the system 10 can emit beams of different wavelengths simultaneously, sequentially, or in an alternating pulsed pattern. Thus, advantageously, in some embodiments, two or more treatment procedures can be performed simultaneously, contemporaneously, or immediately sequentially to improve patient outcomes and to reduce the number of patient follow up procedures.

In some embodiments, the fiber optic output cable 209 can be, but is not limited to, substantially similar to fiber optic relay cables 205. In some embodiments where there is only one laser source and beam combiner 207 is not needed, output cable 209 can be the same cable as relay cable 205. More generally, the fiber optic output cable 209 can be any fiber optic cable capable of transmitting the combined beam emitted by the beam combiner 207 to a fiber optic output. In accordance with various embodiments, the output cable 209 can be formed as a single fiber, can be formed as a plurality of smaller, bundled fibers, or can be formed as two or more closely packed individual fibers for separately transmitting two or more distinct beams having different wavelengths.

More generally, although the relay cables 205 and the output cable 209 are shown herein as being fiber optic cables, it will be apparent in view of this disclosure that any optical pathway capable of directing or transmitting EMR from one or more EMR sources to the beam combiner 207 and from the beam combiner 207 to the treatment area can be used in accordance with various embodiments. For example, in some embodiments, the pathways can be constructed of a series of mirrors for directing the EMR beams.

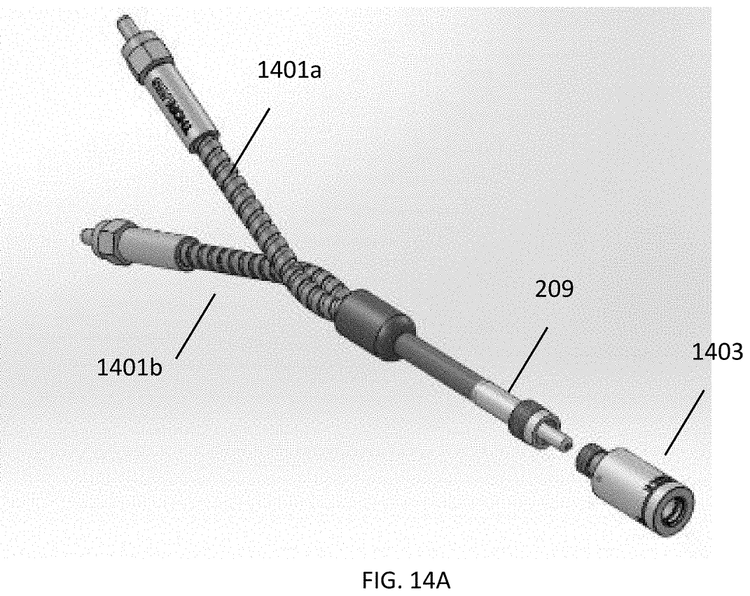

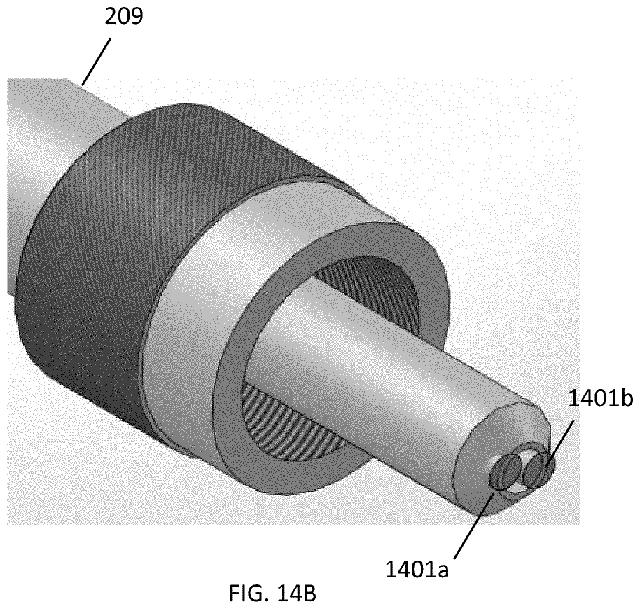

For example, as shown in FIG. 14A, in order to route two separate beams from two distinct EMR sources to a single delivery device (e.g., a hand piece, robotic head, beam shaping optics) 1403, two individual fiber cores 1401a, 1401b can be combined to form a common output cable 209 to direct a beam from each active laser source 203 into a single output fiber connector 211. Referring now to FIG. 14B, because the fiber cores 1401a, 1401b of the common output cable 209 are adjacent and positioned near a center of an optical axis of one or more beam shaping components 1403, the beam shaping components 1403 can produce EMR beam outputs from either or both laser sources 203 with only a slight angular deviation from the true optical axis, the deviation having a negligible effect on beam shape and orientation.

In some embodiments, the fiber optic output cable 209 can also include a fitting 211 positioned at one end thereof for engagement with a device such as a hand piece, robotic head, or other emitter.

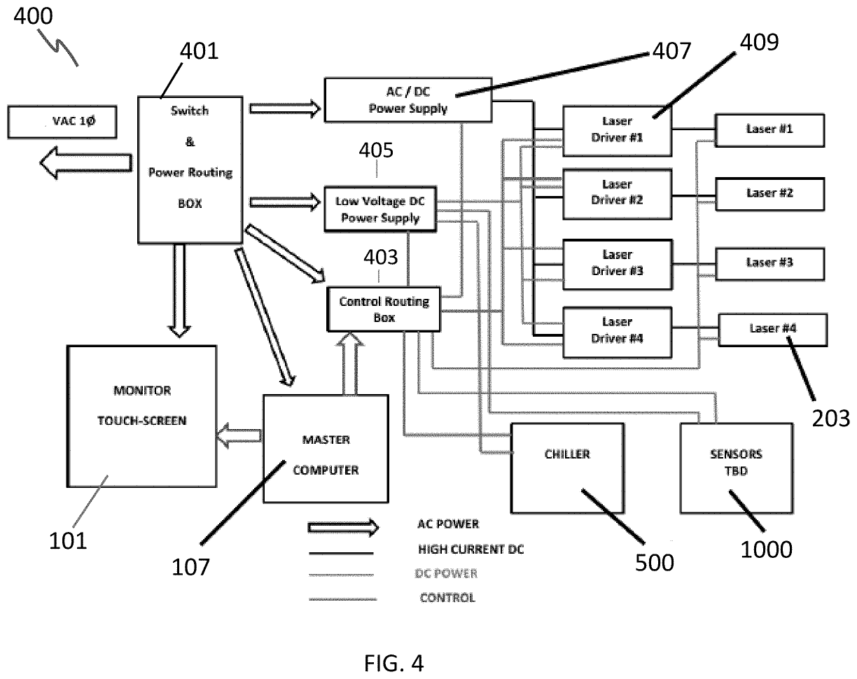

As shown in FIG. 1, in some embodiments, the system 10 can include power and control electronics 400 for powering and controlling various components of the system 10. Referring now to FIG. 4, in some embodiments, power and control electronics 400 can include a switch and power box 401 for receiving AC electrical power from the power cord 103 and distributing AC electrical power to various components as required for operation of the system 10.

The power and control electronics 400 can also include a controller 403, powered by the AC electrical power (e.g., 220 VAC), in electronic communication with the computing device 107 to command one or more additional components of the system 400 to perform one or more directed operations to execute an aesthetic procedure.

The power and control electronics 400 can also include a low voltage ADC 405 for converting AC power from the power box 401 into high or low voltage DC power for operating one or more additional components of the power and control electronics 400. The low voltage ADC 405 can include any suitable ADC, including, for example, a direct conversion ADC, successive approximation ADC, ramp compare ADC, Wilkinson ADC, integrating ADC, delta encoded ADC, pipelined ADC, sigma delta ADC, time interleaved ADC, intermediate FM stage ADC, any other suitable ADC, or combinations thereof.

The system can also include a high voltage ADC 407 for converting AC power from the power box 401 into high or low voltage DC power for operating one or more additional components of the power and control electronics 400. The high voltage ADC 407 can include any suitable ADC, including, for example, a direct conversion ADC, successive approximation ADC, ramp compare ADC, Wilkinson ADC, integrating ADC, delta encoded ADC, pipelined ADC, sigma delta ADC, time interleaved ADC, intermediate FM stage ADC, any other suitable ADC, or combinations thereof.

The power and control electronics 400 can also include a plurality of diode drivers 409 for delivering drive current to the one or more laser sources 203. The diode drivers 409, in some embodiments, can, for example, be semiconductor devices configured to pass a current through a junction region between an n-type semiconductor and a p-type semiconductor. In such configurations, electrons produced by the n-type semiconductor in the presence of a current source such as DC power supply 407 can result in production of photons upon encountering holes of the p-type semiconductor. The photons can oscillate within the junction region, resulting in an optical gain in the junction region. When the current delivered to the semiconductor device exceeds a threshold current, the optical gain can exceed a threshold intensity, causing the photons to exit the junction region as a beam of laser light. In general, after reaching the threshold current, the laser output increases in power density (intensity) linearly in proportion to an increase in the input current. Furthermore, in some embodiments, the diode drivers 409 can also include regulators for controlling current input and one or more protective features such as, for example reverse current blocking and electrical spike suppression features.

In some embodiments, a single DC power supply 407 or 405 can be used for multiple diode drivers if the required compliance voltage for each driver 409/laser source 203 pair is the same and within the limits of the chosen diode driver. Sufficient current capability of the DC power supply 407 or 405 to operate the number of simultaneously driven driver 409/laser source 203 pairs is required. Advantageously, no special switching is required between the DC power supply 407 or 405 and the driver 409 or driver 409 and laser source 203. The DC power supply 407 or 405, in some embodiments, can be parallel connected to each driver 409. This presents an option for multiplexing the main power supply to the multiple laser sources 203.

In such embodiments, each of the diode drivers 409, when activated, can directly drive a single laser source 203 to produce a beam having a particular wavelength as discussed above with reference to FIG. 2. Thus, in some embodiments, one driver 409/laser source 203 pair can be activated for aesthetic procedures requiring a single wavelength EMR beam for treatment. Alternatively, in some embodiments, multiple driver 409/laser source 203 pairs can be activated any of simultaneously, sequentially, or in an alternating pulsed pattern to provide two or more wavelengths as required for a particular treatment and/or to combine or expedite treatments.

Referring again to FIG. 1, the system 10 can also include one or more cooling systems 500 for removing heat produced by the electromagnetic array 200 and the power and control electronics 400 and for delivering cold air for cooling of a patient's skin during a procedure. In general, cooling requirements are primarily dependent on heat generated by the electromagnetic array 200. For example, for a system operating a 100 W EMR source in a small package with an efficiency of about 50%, the cooling capacity can be as low as 200 watts.

Such heat is typically dissipated by one or more of forced air (e.g., fan) cooling, thermoelectric cooling, flowing coolant directly through the electromagnetic array 200, or a cooling plate. While some cooling systems have drawbacks, baseplate cooling to cold plate is efficient, safe, quiet, and compact. Large cold plates can accommodate multiple EMR source heads and drive electronics. In some embodiments, several cold plates can be connected in series to the master circulating chiller. In some embodiments, one or more additional master circulating chillers can be provided as required to accommodate different cooling temperature requirements.

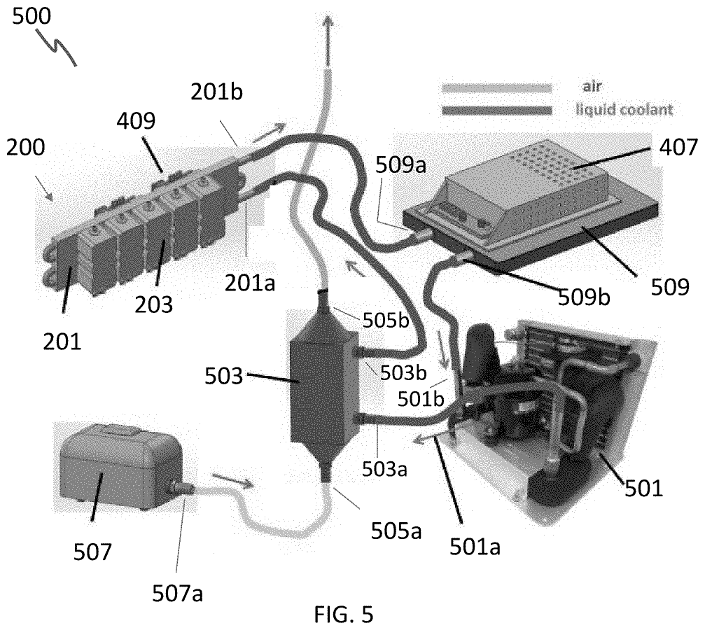

As shown in FIG. 5, the cooling system 500 can include a refrigeration unit 501 such as a refrigerated heat exchanger, thermoelectric cooler, cold water heat exchanger, any other suitable cooling device, or combinations thereof. In some embodiments, a coolant output 501a can exit refrigerated coolant from the refrigeration unit 501. The coolant can then be routed through one or multiple devices to provide cooling and remove heat before being directed to a coolant return 501b for further refrigeration. Although shown having a single refrigeration unit 501 herein, it will be apparent in view of this disclosure that, in some embodiments, the cooling system 500 can include one or more additional independent refrigeration units 501 to cool various components at different temperatures. For example, in some embodiments, a first refrigeration unit can provide coolant at a temperature of about 0° C. to about 5° C. to chill cooling air for impingement, 503, on a patient during a procedure, a second refrigeration unit can provide coolant at a temperature of about 20° C. to about 25° C. to cool the electromagnetic array 200 without generating condensation, which could damage the laser sources 203, and/or a third refrigeration unit can provide coolant at a temperature of about 10° C. to about 30° C. to cool power supply 405 or 407. It will still further be apparent in view of this disclosure that, in some embodiments, the refrigeration unit 501 and/or the cooling system 500 can be provided with a temperature adjustment feature for permitting responsive adjustment of the coolant temperature depending on operational conditions and/or sensor feedback as needed to maintain therapeutically acceptable temperatures in the treatment area consistent with procedure requirements and to maintain operationally acceptable temperatures within the system 10 consistent with equipment requirements.

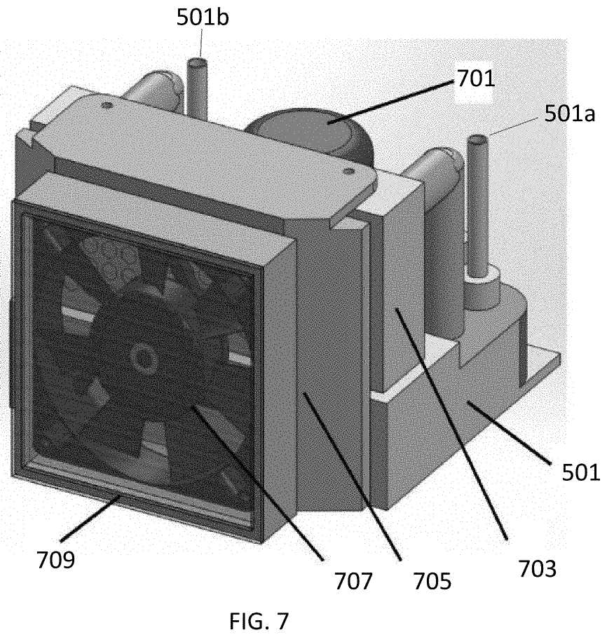

Referring now to FIG. 7, the refrigeration unit 501 can also include a compressor 701, a condenser 703, and an evaporator (not shown). The refrigeration unit 501 can provide forced convection cooling of the condenser 703 through a plenum 705 using a fan 707. In some embodiments, to improve air quality, the plenum 705 and fan 707 can include a HEPA filter 709 to capture particles, bacteria, and viruses, thereby preventing circulation of such particles, bacteria, and viruses through air surrounding the system 10.

As shown in FIG. 5, in some embodiments, the coolant can be directed to a coolant inlet 503a of a heat exchanger 503, flowed through the heat exchanger 503, and exited from the heat exchanger 503 via coolant outlet 503b. The heat exchanger 503 can be any suitable device for cooling air or other gasses driven through the heat exchanger 503 via gas inlet 505a and exited via gas outlet 505b. The air or gas flowing in the heat exchanger 503, in some embodiments, can be used for cooling the skin of a patient during a procedure. For example, in some embodiments, the air or gas can cool the patient skin to a target temperature in the range of 0 to 20° C. via a gas impingement cooling of the skin during the procedure in order to maintain a therapeutically acceptable temperature range.

In some embodiments, the air or gas can be driven through the heat exchanger 503 by a pump 507. The pump 507, in some embodiments, can be any suitable device capable of driving the gas through the heat exchanger 503 and onward to a jet impingement nozzle (not shown). In some embodiments, in order to maintain a therapeutically acceptable temperature at the treatment area (e.g., a patient's skin), the pump 507 can be in electronic communication with the controller 403 to receive instructions from the controller for adjusting a flow rate of the cooling air or gas responsive to feedback from one or more temperature sensors monitoring the treatment area.

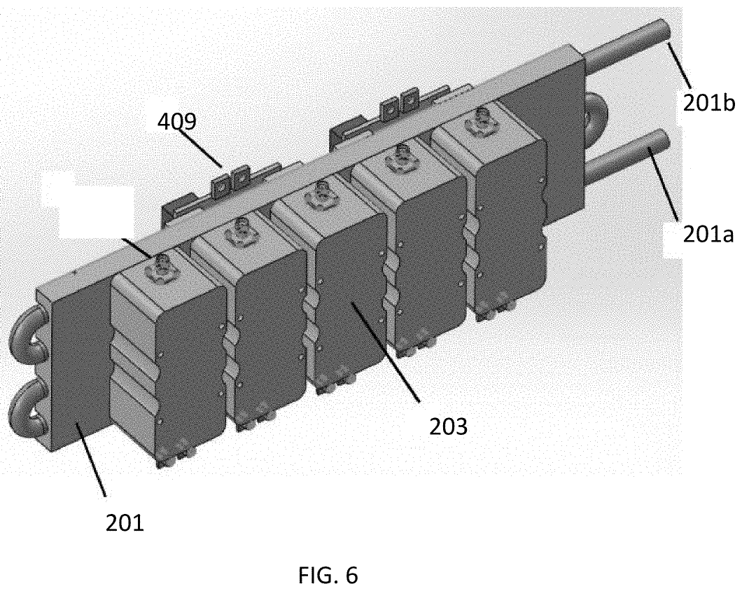

The cooling system 500, in some embodiments, can route the coolant from the coolant outlet 503b of the heat exchanger 503 or directly from outlet 501a of a refrigeration unit to a first coolant port 201a of a mount 201 as described above with reference to FIG. 2. The coolant can chill the mount 201, thereby providing a heat sink for cooling the one or more laser sources 203 mounted to the mount 201. As shown with greater detail in FIG. 6, in some embodiments, the mount 201 can be a cold plate for cooling the laser sources 203 mounted thereto. In some embodiments, the mount 201 can also include one or more of the diode drivers 409 mounted thereto. In such embodiments, the cold plate mount 201 can advantageously cool both the diode drivers 409 and the laser sources 203 with a single cooling mechanism. Although the mount 201 cooling plate is shown herein as being sized for five laser sources 203 and two diode drivers 409, it will be apparent in view of this disclosure that the mount 201 can be sized to accommodate any number or combination of laser sources 203 and diode drivers 409.

Referring again to FIG. 5, the coolant can be exited from the mount 201 via a second coolant port 201b or directly from port 501a of a refrigeration unit and routed to a coolant input 509a of a baseplate 509 of the DC power supply 407 or 405 to provide cooling to the DC power supply 407 or 405. The coolant can be exited from the baseplate 509 via a coolant output 509b of the baseplate 509 and routed to the coolant return 501b of the refrigeration unit 501.

Referring again to FIG. 1, the system 10 can also include one or more positioning apparatus 900 in accordance with various embodiments for permitting movement, control, and positioning of a device 950 (also referred herein as treatment head) coupled to the output cable 209. In some prior art aesthetic EMR devices, they apply EMR energy with stationary or manually manipulated devices. Thus, the application of the heat energy is typically limited to small, fixed areas in the case of stationary devices or, in the case of manually manipulated devices, a relatively uncontrolled and nonuniform dosage of total energy. Accordingly, in some embodiments, the positioning apparatus 900 can provide a multi-axis, computer-controlled mechanism for controlled movement, orientation, and positioning of the device 950 used for emitting the EMR for treatment. In some embodiments, such positioning apparatus 900 can provide movement over a predefined treatment zone. In some embodiments, the computer control provides improved control and movement over stationary or manually operated systems. In particular, computer control can provide for scanning the device 950 across large areas during treatment to provide uniform heating of the target treatment area. For purpose of the describing the present invention, scanning can include controlled movement of a device (e.g., device 950 or another device) over a treatment area. During a scanning operation the device can perform different operations, such as for example, directing energy, performing visual recognition of an area, directing visual indicators, applying cooling, etc. Furthermore, the treatment pattern can be modified to any shape desired for treatment. For example, treatment patterns can be programmed to avoid existing scar tissue or the belly button area.

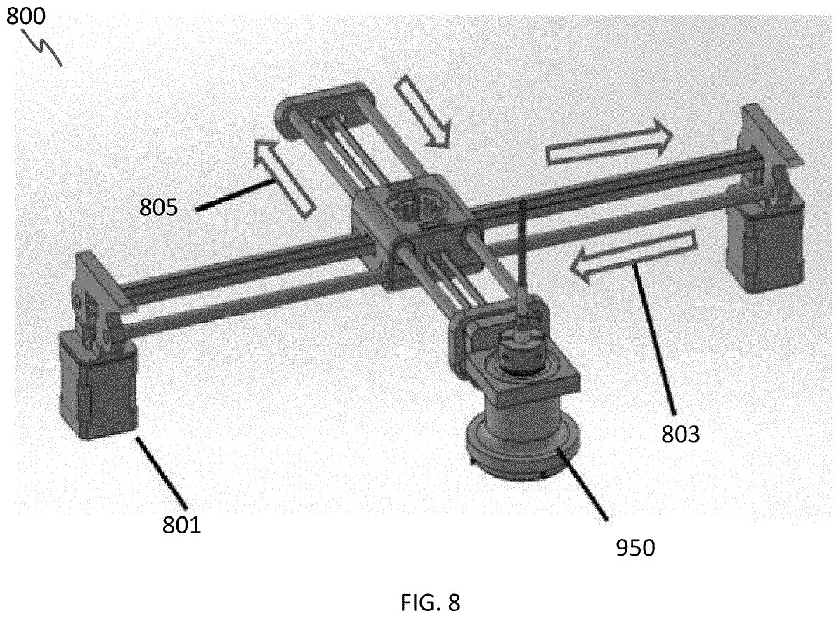

In order to provide desired coverage of an area to be treated and permit proper positioning of the device 950, the positioning apparatus 900 can be provided with any number of degrees of freedom for movement of the device 950. For example, in some cases a treatment process can employ only one DOF and move the device 950 back and forth over the treatment area. As shown in FIG. 8, in some embodiments having a substantially planar target treatment area, the positioning apparatus can be a two degree of freedom control device 800 having a first rail 803 for providing movement along an x-axis of the device 800 and a second rail 805 for providing movement along a y-axis of the device 800.

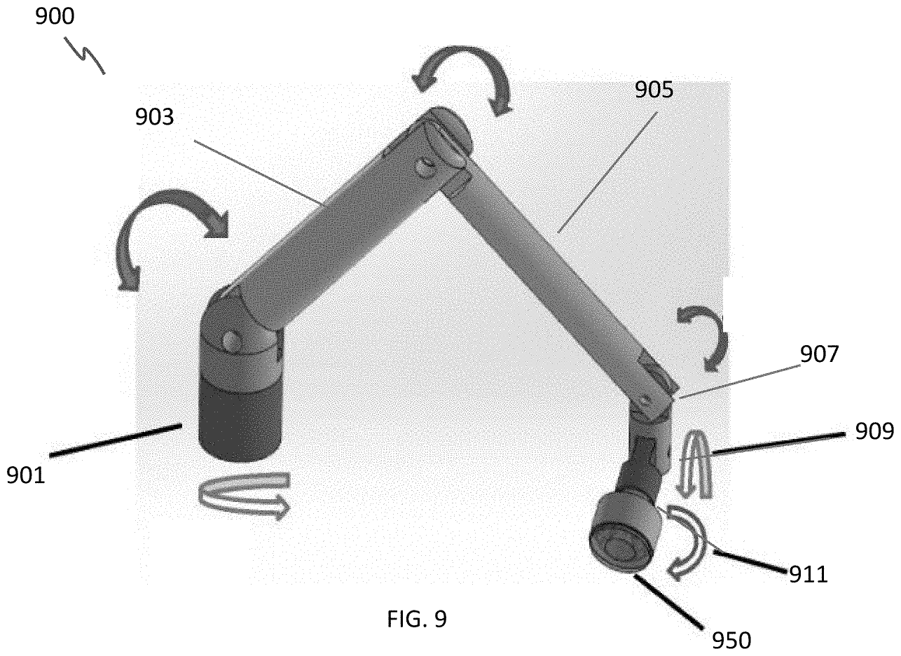

Referring now to FIG. 9, in some embodiments, the positioning apparatus 900 can be a six degree of freedom robotic arm. The positioning apparatus 900 can include, for example, a rotatable base 901 providing a first degree of freedom of rotation of the positioning apparatus 900. The rotatable base 901 can be pivotably engaged with a first segment 903 to provide a second degree of freedom. The first segment 903 can be pivotably engaged with a second segment 905 to provide a third degree of freedom. The second segment 905 can be pivotably engaged with a third segment 907 to provide a fourth degree of freedom. The third segment 907 can be pivotably engaged with a fourth segment 909 to provide a fifth degree of freedom. The fourth segment 909 includes a rotatable portion 911 for rotating the device 950. In general, the rotatable base 901 can be engaged with the housing 100 of the system 10 or can be attached to a separate platform for positioning nearer the target treatment area. The six degrees of freedom of the positioning apparatus 900 can advantageously be used to follow the targeted patient's body shape and match the treatment zone desired.

Such positioning apparatus 900 can be important in various procedures such as, for example, in the case of subcutaneous fat reduction, where deposition of heat into the subcutaneous fat requires reaching and maintaining a therapeutically acceptable temperature range such as, for example, about 40° C. to about 48° C. over a period of time. In particular, in some embodiments, lower temperatures have no fat reduction benefit and higher temperatures can cause severe necrosis, cell damage, and scarring. Conventional devices modulate or cycle the power off and on to maintain this temperature range. However, the low thermal conductivity of fat makes EMR source on/off cycle times compatible with a scanning or moving the device during treatment to cover larger treatment areas and to avoid overheating of the treated tissue. Thus, the positioning apparatus 900 can be programmed to control the device 950 to follow the targeted patient's body shape and match the treatment zone desired. In such embodiments, the heat energy delivered, the treatment area, the dwell time for energy on and the heat source return time to maintain the target temperature are factors that can be used to determine the overall treatment protocol. Patient information, sensors, and feedback can also all be used to maintain a uniform heating over the entire treatment site by scanning the energy delivery module in such a fashion as to cover the entire site. However, it will be apparent in view of this disclosure that, in some embodiments, the system 10 may not include a positioning apparatus 900 and that the device 950 can instead be connected to the housing by the fiber output 209 and/or a cooling air source for manual operation and positioning. It will still further be apparent in view of this disclosure that, in some embodiments, the system 10 may include both a device 950 for use with the positioning apparatus 900 and a manually operated and positioned device 950 for use as required by a particular procedure. For example, the manually operated and positioned device 950 can be used where desired.

Furthermore, sensors 1000 and corresponding sensor feedback can be monitored in real time by the computing device 107 to permit the computing device 107 to reactively instruct (e.g., via controller 403) the positioning apparatus 900 to reposition the device 950. For example, in some embodiments, if the sensors 1000 detect that skin temperature is too high, the computing system 107 can instruct the positioning apparatus 900 to move the device 950 to a new location and/or to scan faster during treatment to reduce dwell time in one area and prevent overheating. In some embodiments, the if the sensors 1000 detect that skin temperature is too low, the computing system 107 can instruct the positioning apparatus 900 to increase a distance or spacing between the device 950 and the target surface to reduce the effects of cooling air flowing through the device 950. Still further, sensors 1000 can be included to detect a position of the device 950 relative to the surface to be treated. In such embodiments, the positioning apparatus 900 can responsively adjust a position or orientation of the device 950 relative to the surface to be treated according to the sensor 1000 feedback. For example, in some embodiments, the positioning apparatus 900 can maintain a prescribed separation height between the device 950 and the surface to be treated.

Numerical simulation modeling for an EMR source in the near-infrared where transmission to the subcutaneous fat is achieved shows that for 1.5 watts per centimeter squared over a 2×2 inch area, the adipose tissue at 12 mm depth reaches 47° C. within 50 seconds. This sample model also included controlled cooling of the skin at 30° C. Simulations show that, without cooling the skin surface would reach an unacceptable temperature of more than 57° C. In this case, the model also shows how the adipose tissue's temperature will decay with time. This model indicates that the patient can be treated in one zone for 50 seconds, after which the robotic control moves the energy source to the next zone for another 50 seconds. This can be repeated to multiple zones, only requiring return to the initial zone before its temperature falls too far below the target temperature range of 40 to 52° C. for efficient hyperthermia apoptosis. Additional modeling studies show that the second treatment duration requires less time to reach the 52° C. temperature and that the reduction in required reheat time is asymptotic. In some instances, as the treatment head is scanning around the pattern loop, it will scan at a particular power level until the tissue reaches the target temperature (52° C.) it can then decrease power until it reaches a plateau in which the decay matches the power level, and the power may not need to be decreased further once plateaued.

In some embodiments of the present invention, the control system can monitor a temperature of the treatment area and then skin within the proximity of the treatment area and can shut down the EMR delivery when the temperature of the skin or treatment area gets too high. For example, when a temperature of an area is outside a predetermined temperature range, the controller can initiate an OFF portion of the duty cycle to allow that area to cool. While in the OFF portion of the duty cycle the affected area can cool naturally or a cooling airflow can be applied by the system to the area. In some embodiments, the skin can be cooled by scanning for a brief time with the EMR delivery shut down while applying cooling air until skin temperature is reduced to the desired level. In some embodiments, instead of remaining at a first treatment location waiting for the skin or treatment area temperature to return to an acceptable range or level, the laser head can be moved to a second treatment location, during the OFF portion of the duty cycle, to begin treatment of the second treatment location, thereby reducing the overall procedure time.

It is important to note that this model is an example based on defined tissue characteristics. However, dwell times and reheat cycles may need to be adjusted on a case by case basis based on, for example, patient skin type, patient characteristics, wavelength, cooling characteristics, etc. Additionally, it will be apparent in view of this disclosure that the treatment does not need to target 52° C. and can instead target a lower temperature within a procedure-specific range. For example, the treatment can be successful with lower target temperatures, such as 44° C. In each case, the patient type and treatment time can be adjusted to a range of target temperatures. Additionally, it will be apparent in view of this disclosure that, in some embodiments, the temperature can be permitted to fall below the minimum effective temperature of 40° C. for short periods of time with reheating applied to raise the temperature back into the hyperthermia apoptosis targeted range. The application of computer control with the appropriate input parameters allows an efficient and optimized treatment protocol.

In some embodiments, a pattern may be scanned in which the energy source returns to the initial treatment site in a time equal to the expected decay time of the temperature. Since reheating to the target temperature requires less time on the second pass, the energy source may be moved at a faster rate on the second pass over tissues. Energy source scanning patterns may be optimized for treatment of a maximum area in a minimum time and will depend upon patient anatomy and tissue parameters. Scan rates and treatment patterns may be modified in real time based upon measured skin temperatures and heat flux and predicted subcutaneous tissue temperature. Energy source power may be modulated during movement of the energy source to further optimize treatment.

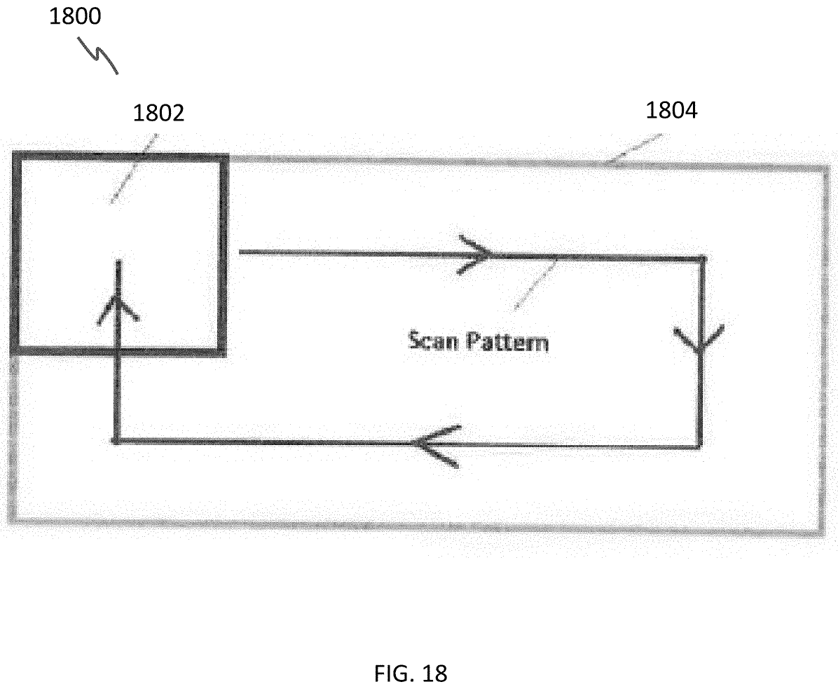

Referring to FIG. 18, in some embodiments, the laser head can be continuously scanned over a region or treatment zone 1800 of target tissue that has an area that is larger than the cross-sectional area 1802 of the laser beam itself. In such instances a scan pattern 1804 can be created such that the cross-sectional area 1802 of the laser beam (or EMR beam) can be applied in a non-overlapping manner. For example, the beam can be swept over the target region in a pattern 1804 that returns the beam to its starting point. In some embodiments, the scan pattern 1804 can be repeated until tissue has dwelled in the therapeutic temperature range for a time adequate for apoptosis. In such embodiments, laser power can be increased to a level that keeps the average power density (from the beam) seen by tissue more than the power density needed for apoptosis when the laser beam is stationary.

For example, the cross-sectional area of the laser beam can be 4.3 cm by 4.3 cm or about 18.5 cm², and this square cross section can be scanned in a 4×2 pattern, or an area that is 4×4.3 cm by 2×4.3 cm for a total area of tissue in the scan region of 148 cm². Continuing the example, in some embodiments, the beam can be scanned over the two rows of tissue and may be turned off during the transitions between rows. As the beam is repeatedly scanned over the region, the fraction of time that a given 18.5 cm² of tissue is in the beam is 18.5/148=1/8=0.125. Given a laser power during the initial scan of 150 Watts, the instantaneous power density is equal to 150/18.5=8.1 Watts/cm². The average laser power density delivered to any tissue during the initial scan over the target tissue region is then equal to 8.1/8 Watts/cm²=1.01 Watts/cm². Increasing the laser power from a therapeutic level of 1.01 Watts/cm² for a stationary beam to 1.01/0.125=8.1 Watts/cm² for the moving beam keeps the average power in any part of the scanned region at 1.01 Watts/cm². In this example, any given tissue within the treatment area is in the 8.1 Watt/cm² laser beam for 12.5% of the time, and out of the beam for 87.5% of the time (i.e., a 12.5% duty cycle). During the time that the tissue is out of the beam its temperature drops but remains in the therapeutic range. The laser is always ON in this embodiment, and the desired duty cycle can be achieved by scanning the beam at a particular rate and/or pattern. The average power density never exceeds a critical safety value of about 1.5 Watts/cm². In some embodiments, a critical safety value is more than 5 W/cm².

In some embodiments, a pattern 1804 can be created to create a rectangular treatment zone 1800 or treatment area, as depicted in FIG. 18. The treatment zone 1800 can have a length and a width, with the length being approximately a whole number multiple of the length of the laser beam and the width of the cross-sectional area 1802 of the beam being approximately a whole number multiple of the width of the laser beam. For example, as depicted in FIG. 18, the perimeter of the scan line can be equal to eight times the side dimension of the square laser beam. Continuing the example, if the laser beam cross section side dimension is 4.3 cm, then the scan perimeter is 34.4 cm. As would be appreciated by one skilled in the art, the treatment zone 1800 can include any combination of sizes, shapes, and patterns to provide a non-overlapping application of the laser beam within the treatment area. For example, the treatment area can have a perimeter of four times the side dimension of the square laser beam. In alternative embodiments, the sizes, shapes, and patterns for a treatment zone 1800 can be specifically designed to have overlapping while maintaining a consistent target temperature range through the treatment area. Similarly, the sizes, shapes, and patterns for a treatment zone 1800 can be specifically designed to facilitate operation with a non-square laser beam, for example, a circular laser beam.

Continuing with FIG. 18, in some embodiments, scanning mode exposes tissue to a duty cycle that heats the entire treatment zone 1800 to the target temperature range. Continuing the above example, one complete scan can be completed in about 4 seconds, such that the laser scan speed is 8.6 cm/sec, and tissue is in the path of the laser beam for the duty cycle of ⅛ or for 0.5 seconds on each scan of the target tissue. The lower thermal conductivity of the adipose tissue helps maintain our target temperature range during scanning. For a given laser power density, in some embodiments, tissue models can be used to predict the maximum temperature that will be reached during the time that the laser is over a given tissue, and the minimum temperature reached while the laser is not over this given tissue. For example, the model can evaluate a wattage/power coming out of the laser beam and the temperature/flow rate of any cooling air to calculate subcutaneous temperature to determine what skin temperature should be. The model can also compensate changes over time to adjust wattage/power of the laser beam to maintain proper temperature zone for the subcutaneous tissue. A maximum dwell time, or given the dimension of the laser beam, a minimum scan rate is required to prevent tissue overheating, and a minimum scan rate is required to prevent excess cooling during the time the laser beam is not directed at a given tissue. In some examples the maximum dwell time is in the range of 0.25 to 1.0 second, and given a 4.3 cm square laser beam, the minimum scan rate is in the range of 4.3 cm/sec to 17 cm/sec for a laser power density of 8.1 Watts/cm². In some embodiments one or more sensors can be used to verify the model and provide input for any corrections. The one or more sensors can also be used for safety purposes to ensure that a predetermined temperature is not exceeded.

To ensure apoptosis of all tissue in the target region, the tissue must be held in the target temperature range for a time adequate to denature its cells. It has been determined that an exposure time of 15 minutes is adequate to cause apoptosis in tissue that is held in the target range of 40° C.-52° C. Since heat is retained in the target region, the average power needed to keep the temperature in the target range decreases with time. In some embodiments, a decrease in average power may be achieved by making reductions in the laser power density over the treatment duration. For example, tissue modelling has shown that about 50 scans of the tissue region of FIG. 18 with laser power set at 150 Watts and a square laser beam cross section 4.3 cm on a side can bring tissue to the high end of the target temperature range, in 200 seconds or 3⅓ minutes. Thereafter, tissue can be maintained in the target temperature range when the laser power is reduced to 130 Watts for 15 scans or one minute, then to 115 Watts for 15 scans or one minute, then to 100 Watts for one minute, and finally to 85 Watts for 130 scans or 8⅔ minutes, for a total treatment time of 15 minutes.

In some embodiments, it may be possible to raise the temperature of the target tissue (for example, the fat layer for fat reduction procedures) higher than the range of 42° C.-51° C. For some patients, this range is selected in order to keep the skin temperature less than 40-43° C., the temperature where some patients feel pain. In some embodiments, it is possible to raise the temperature of the target tissue to a higher temperature of about 50° C. or about 55° C. without causing pain for the patient. This higher temperature may be used when the heat transfer from the treatment tissue to the skin is low and/or in conjunction with more aggressive skin cooling. If these higher temperatures are used, the treatment time may be reduced from 15 min to about 10 min or to about 5 min.

In some embodiments, a 150 W laser may be used to generate the laser beam (or EMR beam). In this embodiment, the laser can be used to heat the treatment zone 1800 to a temperature where the patient's skin is within an acceptable range. The laser may then be shut off for a period of time, for example, about 5 seconds, about 10 seconds, about 15 seconds, or whatever length of time is necessary to allow to prevent the temperature of the skin from going above an acceptable level while maintaining the temperature of the subcutaneous treatment area at an acceptable level. In some further embodiments, the laser can be moved to a new treatment area while the user/physician is waiting for the first treatment area to cool. In embodiments such as this, the higher laser power will heat the subcutaneous tissue faster than a lower power laser and the time that the laser is off or moved to another treatment area will allow the surface tissue to cool while maintaining a high temperature in the treatment area.

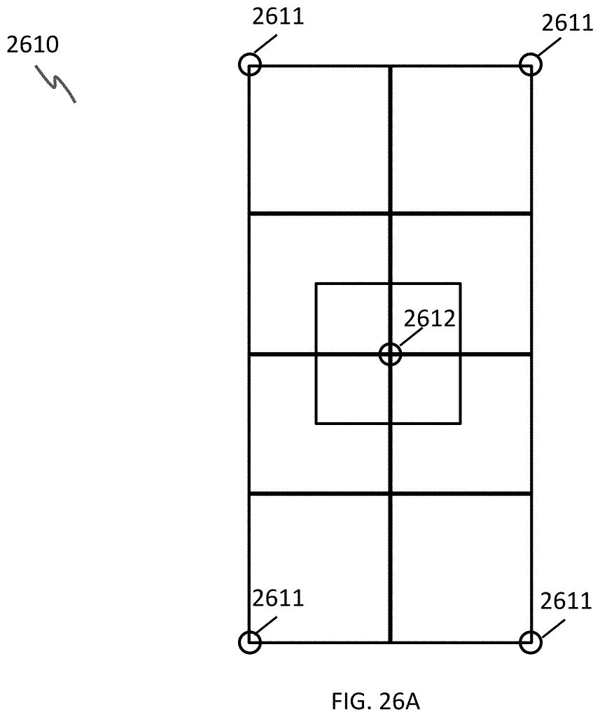

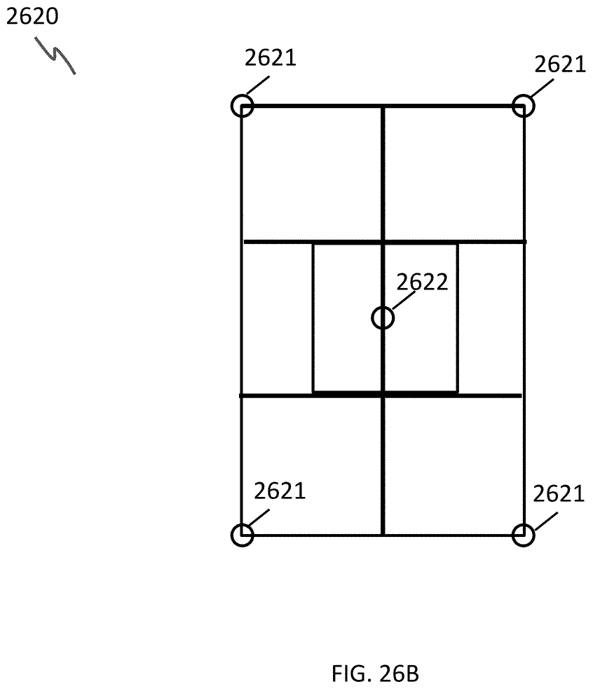

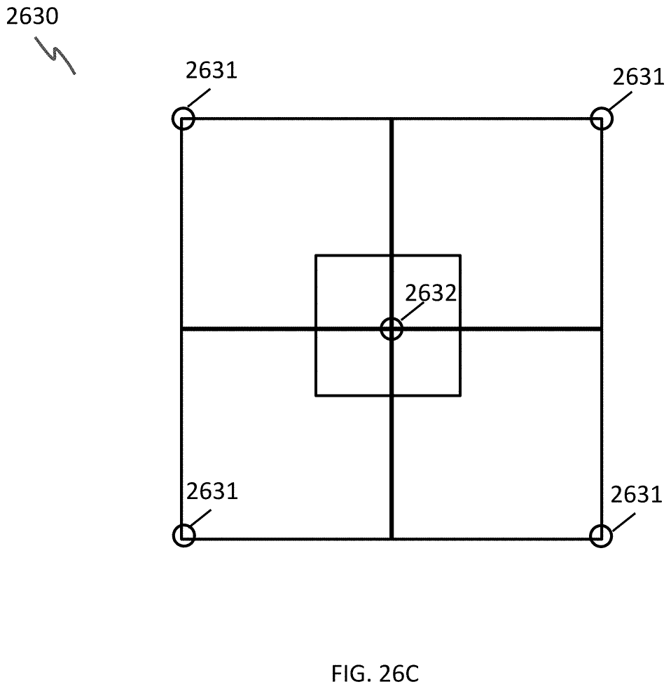

In some embodiments, a treatment zone, treatment area, or target tissue region can be created, in part, by using a template. For example, a template as shown in FIG. 26A, 26B, or 26C can be used to mark the treatment zone 1800 of the shape shown in FIG. 18. The templates can be any combination of materials, for example, paper, plastic, etc. Templates can be used by a physician, or other user, to assist in registering or mapping a treatment area on a patient for use by the treatment device. The shape and dimensions of the template can be used to indicate to the system the boundaries of the treatment area, as discussed in greater detail herein. The physician can be provided with different predetermined template with preset shapes, sizes, etc. and can select appropriate template for the desired treatment. The physician can select a template that will sufficiently fit and cover a desired treatment area on a patient. The patient will be placed in a position adjacent to the treatment system and physician can place a selected template on the area of the patient's body that is to be treated. The selected template can be placed in different orientations and can be rotates to fit the desired treatment area. In some embodiments, a physician can create a custom template to fit a particular treatment area.

In some embodiments, the template can be provided to assist in creating identifiable markings that are readable as inputs by the system for alignment of the treatment head, arm, etc. of the treatment device. The markings can be used to indicate where the boundaries of the treatment area should be and the treatment device can create a treatment pattern based on those boundaries. In some embodiments, with a template in place, the physician can place markings as indicated by the template on the patient's body. For example, using a template 2610, 2620, or 2630 as shown in FIG. 26A, 26B, or 26C the markings can be created at each of the four corners of a given template 2610, 2620, or 2630. The markings can be created using any combination of methods that are machine readable. For example, the markings can be made on the skin with a dark marker, a UV marker, etc. that are machine detectable/readable using any combination of camera recognition, imaging, etc. With the markings in place, the physician can remove the template, and move the arm over the marked area (manually or automatically). For example, the physician can manually move the arm using mechanical or electronic user interface buttons on the user interface (101 of FIG. 1) or a joystick or other device can be used to manually move the treatment arm. Once the arm is over the proposed treatment area, the location of the markings can be identified, set, and/or saved the for use during treatment by the system. In some embodiments, the treatment area can be registered by moving the aesthetic treatment device over each the markings, which when identified can be recorded by the system as datums. The recorded markings can then be used by the system to create an outline or map the treatment area. For example, the system can create boundary lines between each of the markings in the order that the markers where registered with the device (e.g., order in which a user designated by moving the device over the markings) and connecting the last marking with the first marking to create a continuous outline. In some embodiments, an outline can be associated with a particular template, for example, via user in put or scanning of a code on the template such that the markings provide the orientation of the preset outline designated by the template. Regardless of the creation of the outline, the outline can be used to map the treatment area and create a pattern for directing the laser beam (or EMR beam) over the treatment area. In some embodiments, the laser 107 can be used by the physician user to mark the corner or boundaries of the treatment area. For example, the physician can move the treatment head over a marking, as reflected by the visible beam of the laser and then register the marking with the system (e.g., hitting a button) to create the boundary. In some embodiments, the laser 107 can also provide visual confirm that machine is treating area that the user wants to be treated.

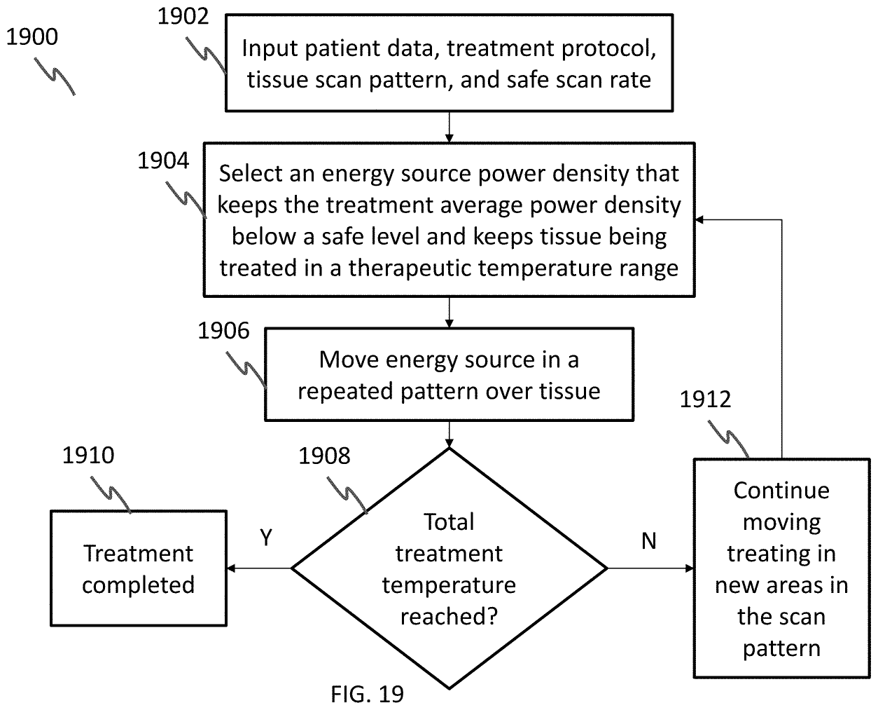

Referring now to FIG. 19, an example process 1900 for aesthetic treatment of a region of tissue on a patient is shown. The process 1900 can be implemented using any combination of devices and systems discussed with respect to FIGS. 1-17 and 20-25. At step 1902, patient data can be input and an appropriate scan pattern and treatment protocol are selected. The patient data can include any combination of data that may be relevant to the treatment process. For example, patient data can include age, race, gender, weight, body mass index (BMI), skin tone, etc. The appropriate scan pattern and treatment protocol can be provided by the system based on any combination of information. For example, the appropriate scan pattern and treatment protocol can be selected from a list of available options based on the user input data. Examples of treatment protocol inputs can include desired beam power, procedure type, wavelength or wavelengths to be applied, pulse duration, treatment duration, beam pattern, treatment area temperature, therapy parameters, skin temperature data (generic or patient specific), skin temperature heat flux data (generic or patient specific), timing data, etc. Thereafter, a safe scan rate can be input, which can be based on the previously input data. Some other inputs can include the physician picking template to cover an area for treatment or mark or modify an area not to be treated. For example, if the treatment area includes a scar that may absorb heat faster than other tissue, a physician can place a block over the scar to reflect the laser beam to avoid overheating. In some embodiments, the user can select power level (e.g., full power or light power) based on a desired procedure or result. For example, full power could be used for larger fat reduction and light power for lower fat reduction.

At step 1904, an energy source power density that keeps the treatment average power density below a predetermined safe level and keeps the tissue in a therapeutic temperate range is selected. The energy source power density can be set to a level that keeps the power density averaged over the entire treatment time less than a critical value. The options for the energy source power density can be recommended by the system based on the inputs from step 1902. In some embodiments, a higher laser power density can be used during a procedure because the laser beam is always moving, such that each tissue may see a lower amount of energy. For example, application of a 150-watt laser over a 4.3×4.3 cm area would be 8.1 W/cm² which may be too high if stationary. However, by scanning over a 8.6×17.2 cm area, the average power is 1 W/cm² and with efficient skin cooling appropriate in maintaining comfortable and safe skin temperature but reaching the target high fat temperatures. These values can be adjusted for equivalent average power density.

The inputs in steps 1902 and 1904 can be either entered by the user or stored or calculated by the treatment system. For example, the physician can use a system 2000 or similar to provide the inputs used to determine the laser scan pattern and scan speed. The system can also be used to provide feedback to the user, for example, laser power and temperature measurements can be shown on the display as the robot arm scans the laser head.

At step 1906, the energy source is moved over the first treatment area in the scan pattern and treatment begins as the energy source scans. For example, the energy source can be moved over treatment zone 1800 of FIG. 18. The scan pattern is completed and the energy source returns to its starting point.

At step 1908, the system can check to determine whether a total treatment time has been reached. The total treatment time can be based on predetermined values 15-25 minutes, 20 minutes on average, or it could be based on feedback received form the device. The higher fat temperatures can be more effective and a treatment for 20 minutes approaches an asymptotic level. In some embodiments, the energy can be gradually reduced to maintain the target temperature throughout the total treatment time. The operator can select lower levels and also has the option of manually selected quick cools where the laser is off but cooling on for short cycles. If the total treatment time has been reached, then the process 1900 will advance to step 1910.

At step 1910, when the total treatment time has been achieved the energy source is turned OFF and the treatment is complete. In some embodiments, once the treatment is completed in a first area, the energy source can be moved to a next treatment area. If the total treatment time has not been reached, then the process 1900 will advance to step 1912. At step 1912, the energy source returns to step 1902 and continues to scan over the tissue in the prescribed pattern. In some embodiments, the controller can automatically adjust the energy being applied by the laser beam based on a combination of power being applied, time spent, movement speed of the treatment head, etc. In some embodiments, the user can also manually intervene to provide adjustment to the energy levels. For example, the user can turn off the laser beam while applying cooling if patient is in discomfort.

In some embodiments the energy source can be a laser beam having a cross sectional area. In some embodiments the laser power density can be in the range of 5 Watts/cm² to 10 Watts/cm². In some embodiments, the total area of the treated region is in the range of 20 cm² to 200 cm². The laser power density averaged over the entire treatment region can be held less than a critical value. In some embodiments the critical averaged laser power density can be 1.5 Watts/cm².

Referring back to FIG. 1, the device 950, in some embodiments, can be configured to emit the combined beam emitted by the beam combiner 207 and received via the fiber output 209 for treatment of the patient. In some embodiments, one or more devices 950 can be interchangeably engageable with the fitting 211 of the fiber optic output cable 209. In general, the device 950 can include mirrors, beam shaping optics or any other appropriate optical elements. For example, the fiber output can be emitted directly on the patient or mated to a collimating device. In a similar fashion, two or more EMR beams can be combined in free space using mirrors and beam splitting optics. The desired beam shape or pattern on the patient can be modified with an optical element, which can be a lens, lens array, a diffractive or refractive beam shaper, or any engineered diffusing device. The resulting beam shape can match the desired treatment pattern. In some embodiments, the output beam can be adjusted to match the desired beam diameter, power level, and be collimated, diverging, or converging. As stated above, one or more of the laser sources 203 can be operated simultaneously, alternately, or in sequences. This can be controlled by the input to each laser source 203 since the fiber cables and routing optics are passive devices. EMR beam switches or interlocks can be included as required for safety and regulation compliance. In some embodiments, the device 950 can also include a distance sensor for providing feedback to the computer 107 for adjusting positioning by the positioning apparatus 900.

Additionally, although shown in FIG. 1 and described herein as being mounted and/or coupled to the positioning apparatus 900, it will be apparent in view of this disclosure that, in some embodiments, the device 950 may, in some embodiments, be used as a manual hand piece. In such embodiments, the device 950 may not be coupled to any positioning apparatus and instead can be coupled to the housing 100 only by the fiber output 209 and/or a cooling air supply for permitting manual operation and positioning of the device 950.

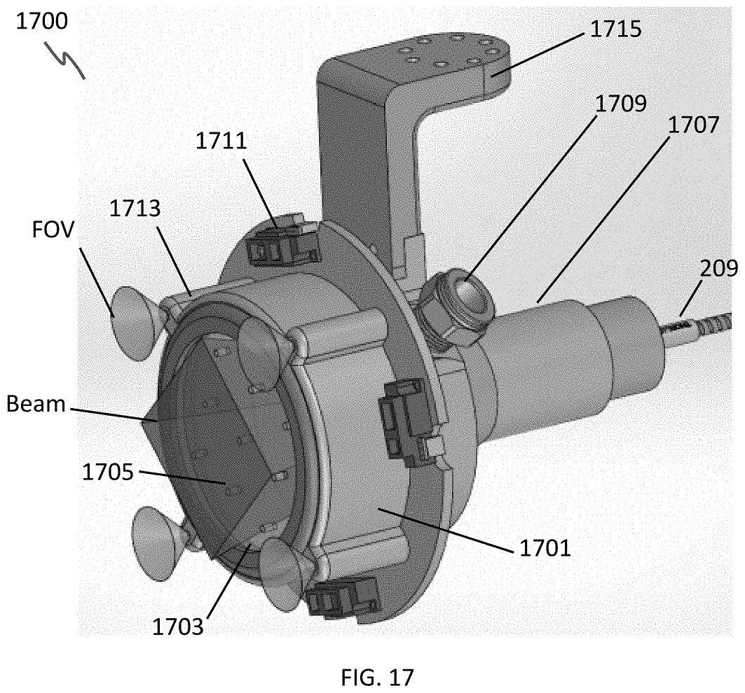

Referring now to FIG. 17, a device 1700 is configured for emitting the EMR beam received via the fiber output 209 for treatment of the patient without contacting the treatment area. In particular, the device 1700 can be configured to direct the EMR beam onto the treatment area, direct cooling airflow onto the treatment area, and provide sensor feedback associated with the treatment area to the controller 403 without the device 1700 or other components of the system making contact with a surface of the treatment area.

To that end, the device 1700 can include a housing 1701 having a surface 1703 to be directed at a treatment area. In order to retain an appropriate shape for airflow control and withstand stresses and forces associated with operation, the housing 1701, in some embodiments, can be constructed of any suitable material such as metals, plastics, transparent plastics, glass, polycarbonates, polymers, sapphire, any other suitable material, or combinations thereof. To the extent that it is desirable to permit the EMR to be transmitted through the housing 1701 to be directed to the treatment area, it may be advantageous to form at least a portion of the housing 1701, in particular at least a portion of the surface 1703, from an optically transparent material. In some embodiments, the entire housing 1701 can be optically transparent. As shown in FIG. 17, in some embodiments, the housing 1701 may not be optically transparent while the surface 1703 is transparent. However, in general, portions of the surface 1703 proximate to or coincident with the EMR beam should generally be optically transparent so as not to interfere with transmission of the EMR.

To facilitate transmission of the EMR beam therethrough, the housing 1701 can also include an EMR port 1707 for engagement with the fiber output 209 to direct the EMR beam through the housing 1701, including the surface 1703, and onto the treatment area. In accordance with various embodiments, the EMR port 1707 can include any fitting capable of engaging the fiber output 209, such as, for example, a Luer slip, a Luer lock, a fitting, a fiber coupler, or any other suitable fitting. More generally, the EMR port 1707 can include any configuration suitable for directing an EMR beam generated by the fiber output 209 through the housing and toward the treatment area.

In some embodiments, the device 1700 can include beam shaping optics (not shown) for producing a particular beam shape. For example, as shown in FIG. 17, the beam shape can be an expanding square beam. However, although the EMR is shown in FIG. 17 as being an expanding square beam, it will be apparent in view of this disclosure that any other beam shape can be used in accordance with various embodiments, including, for example, expanding, converging, straight, homogenized, collimated, circular, square, rectangular, pentagonal, hexagonal, oval, any other suitable shape, or combinations thereof.

The device 1700, as shown in FIG. 17, can also serve as an air-cooling apparatus for cooling the treatment area. To that end, the device 1700 can include one or more cold air ports 1709 for receiving airflow into the housing 1701. Each cold air port 1709 can be any suitable design, size, or shape for connecting to an airflow source, including, for example, an opening in the housing 1701, a tube in fluid communication with the housing, a Luer lock connector, a Luer slip connector, a fitting, any other suitable design, or combinations thereof. In some embodiments, the cold air port 1709 can be formed integrally with the housing 1701. In some embodiments, the cold air port 1709 can be a separate element attached to, fastened to, or otherwise in fluid communication with the housing 1701.

The airflow received into the housing 1701 via the cold air port 1709 can be directed through the surface 1703 toward the treatment area for direct air cooling of the treatment area. In particular, the surface 1703 can include a plurality of openings 1705 formed in the surface 1703 for directing airflow onto the treatment area. In some embodiments, the openings 1705 can be positioned to direct the airflow onto the treatment area at temperatures, flow rates, and exit flow velocities suitable to maintain the treatment area at a therapeutically acceptable temperature range while avoiding interference with the EMR being directed at the treatment area. To that end, openings 1705 coincident with or within close proximity to a portion of the surface 1703 through which the EMR is transmitted (EMR transmission region) can be formed from optically transparent material. To the extent that other openings 1705 are not aligned with the EMR transmission region, those openings may not need to be transparent.

In some embodiments, the plurality of openings 1705 can be arranged in a pattern that can provide substantially uniform cooling over at least the treatment area illuminated by the EMR. In some embodiments, the substantially uniform cooling can extend over an area larger than the treatment area. In such embodiments, pre and post cooling to the treatment area is permitted as the device 1700 is moved from one treatment area to another by the positioning apparatus 900, whether manually or by automated control by the controller 403 as programmed to deliver the appropriate energy to maintain the target temperature range for a procedure.

In order to promote a uniform flow and maintain a desired cooling rate, during use, the openings 1705 can be spaced apart from the target surface to maintain the substantially uniform cooling and to promote efficient jet impingement cooling. For example, in some embodiments, the spacing between the exit plane of the openings 1705 and the target surface can be maintained between zero (0) inches to more than an inch. In some embodiments, the spacing can be about 0.5 inches. More generally, any spacing between the openings 1705 and the target surface can be used so long as substantially uniform cooling can be provided to the treatment area to maintain a therapeutically acceptable temperature range. In general, in jet impingement cooling or impingement cooling, cold or chilled high velocity air can be used to establish a very thin boundary layer that efficiently extracts heat from the treatment surface or skin. In other words, the target velocity can be high enough for impingement cooling on the tissue surface where a thin boundary layer establishes heat extraction that can be 3-4 times greater than that from forced convection. This enables the device 950 to apply a higher laser power. The high velocity air can be provided by forcing a large volume of air through a plurality of openings (e.g., openings 1705) within the treatment head of the device. For example, the velocity range can be greater than 50 m/s. In order to ensure that impingement cooling is happening, the proper air velocity and the proper distance of device 950 above the treatment surface must be chosen and maintained.

The spacing and positioning of the device 1700 can generally be maintained by adjustment of the positioning apparatus 900 as described above with reference to FIG. 9. To facilitate positioning of the device 1700 by the positioning apparatus 900, the device 1700, in some embodiments, can include a device mount 1715 for operatively engaging the device 1700 with the positioning apparatus 900 (not shown in FIG. 17). For example, as shown in FIG. 17, the device mount 1715 can include a flange for removable engagement with the positioning apparatus 900. However, it will be apparent in view of this disclosure that any device mount 1715 capable of providing removable engagement with the positioning apparatus 900 can be used in accordance with various embodiments.

Although shown in FIG. 17 and described herein as including a device mount 1715 and as being mounted to the positioning apparatus 900, it will be apparent in view of this disclosure that, in some embodiments, the device 1700 may, in some embodiments, be used as a manual hand piece. In such embodiments, the device 1700 may not include a device mount 1715 and instead can be coupled to the housing 100 only by the fiber output 209 at the EMR port and/or a cooling air supply at the cold air port 1709 for permitting manual operation and positioning of the device 1700.

In particular, the spacing can be maintained by providing program instructions for the computing device 107 and the controller 403 for operating the positioning apparatus 900 responsive to real time feedback from one or more position sensors 1711 mounted to the housing 1701 and directed toward the treatment area. The position sensors 1711 can be configured to detect one or more of a distance between the device 1700 and the target area, an orientation of the device 1700 relative to the target area, and a position of the device 1700 on the target area. The position sensors 1711 can generally be any suitable sensor for providing non-contact detection of a position of the device 1700 relative to the target area. For example, as shown in FIG. 17, the position sensors 1711 can be infrared location sensors.

In order to aid in meeting procedure requirements, in some embodiments, the device 1700 can include one or more temperature sensors 1713 to provide real time monitoring of a temperature of the treatment area. In particular, as shown in FIG. 17, the temperature sensors 1713 can include one or more non-contact pyrometers to provide non-contact temperature monitoring of the treatment area. In some embodiments, the temperature sensors 1713 can be configured to provide real time temperature feedback to the computer 107 and/or the controller 403. The computer 107 and/or the controller 403 can then responsively adjust one or more operating parameters of the system 10 to maintain the target area at a therapeutically acceptable temperature. For example, in some embodiments, responsive to the temperature feedback provided by the temperature sensors 1713, the controller 403 can at least one of instruct the positioning apparatus 900 to adjust a spacing between the treatment area and the device 1700, instruct the positioning apparatus 900 to adjust a scanning velocity of the emitted EMR beam relative to the target area, instruct the pump 507 to adjust a flow rate of the cooling air or gas, instruct the refrigeration unit 501 to adjust a coolant temperature, thereby adjusting a temperature of the cooling air or gas, instruct the laser sources 203 to adjust a power of the emitted EMR beam(s), shut off or activate one or more of the laser sources 203, instruct the device 1700 to adjust beam shaping optics to alter a beam shape of the emitted EMR beam, or combinations thereof.

While FIG. 17 and other embodiments discussed herein have surface 1703 with openings 1705 through which air can be provided, some embodiments do not have surface 1703. In these embodiments, air flow may be directed to the treatment area via nozzles or other mechanisms for directing air flow. The present invention can use any combination of cooling source and output without departing from scope of the present invention.

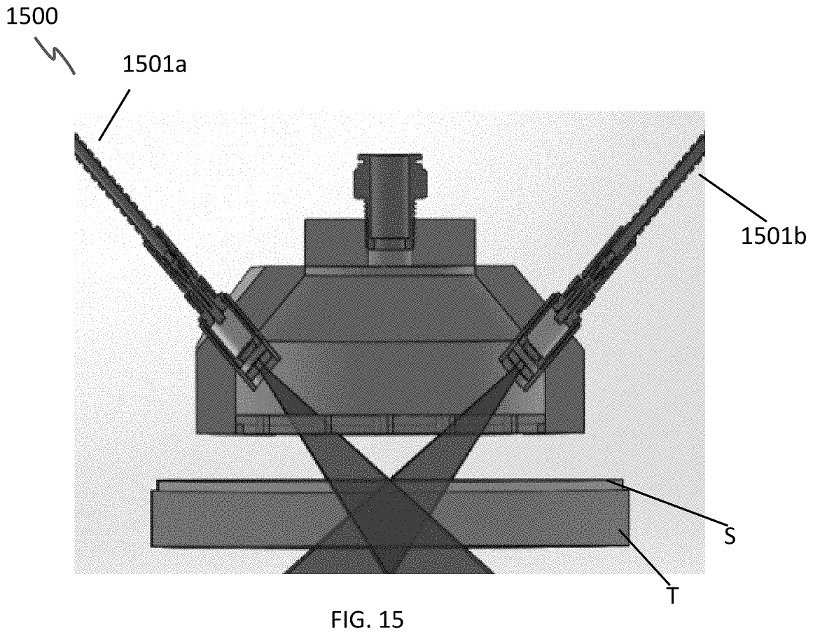

Referring now to FIG. 15, a device 1500 is illustrated wherein the common output cable 209 is split by a beam splitter (not shown) to provide two or more output cables 1501a, 1501b for emitting two or more beams, each delivering only a portion of the total EMR power transmitted by the common output cable 209. Alternatively, in some embodiments, rather than splitting a common output cable 209, the two or more output cables 1501a, 1501b can each be separate, unsplit output cables directly connected to a single laser source 203 and/or the combiner 207. In such embodiments, the array 200 can include a corresponding number of laser sources 203 each having a same wavelength to deliver beams having the same wavelength via each of the emitter cables 1501a, 1501b. Advantageously, such embodiments can permit the use of smaller, lower power, less expensive laser sources 203 because each emitter cable 1501a, 1501b is only required to deliver a portion of the total EMR power used for treatment of the treatment area.

The device 1500 is configured to direct the beams emitted from the output cables 1501a, 1501b at an angle such that the beams impinge separately on a surface to be illuminated S and overlap beneath the surface S in a subsurface tissue to be treated T. Such embodiments can generally provide a lower power density at the point of impingement on the surface S and a higher power density in the overlap region in the tissue T. In particular, power density in the overlap region will scale proportionally with the number of EMR output cables 1501a, 1501b, the power of each EMR beam, and the beam size of each beam in the overlap region. Accordingly, it will be apparent in view of this disclosure that any number of output cables producing any number of EMR beams can be used in accordance with various embodiments as desired to provide a desired power density at the surface S and in the overlap region of the tissue T. For example, in some embodiments, four beams can be provided wherein two pair of opposing beams can be configured in a square arrangement to emit beams at the slant angle to project a rectangular pattern onto the surface S and into the tissue T. In some embodiments, to overlap two more EMR beams from opposing but orthogonal locations, each beam footprint can be rectangular to create a similar projected beam footprint on the treatment plane. More generally, the beam shape of each EMR beam, in some embodiments, can, for example, be diverging, collimated, converging circular, square, rectangular, any other suitable shape, or combinations thereof.

Such a configuration is advantageous because, during, for example, a procedure for hyperthermia of adipose tissue to create apoptosis, the objective is to reach temperatures in the fat (adipose) tissue roughly from 40° C. to 52° C. During this process where the fat tissue is positioned beneath the skin and epidermis by approximately 2.8 mm, the skin, including the active nerve endings therein, can reach temperatures that feel warm or even hot to the patient. Although cold air or cryogenic cooling is typically provided, higher EMR power densities may nevertheless raise skin temperature to an uncomfortable temperature. In such cases, splitting the EMR power into two or more beams impinging separately on the surface of the skin can reduce local skin heating. On the other hand, the sum power of all overlapping beams is concentrated where the EMR beams overlap. Because maximum power is achieved in the overlap region, higher temperatures can be achieved in the overlap region for more efficient apoptosis. Conversely, the lower power density on the skin, epidermis, and dermis will result in lower temperatures in those regions. In some embodiments, such lower power density can reduce skin cooling requirements for maintaining patient comfort and safety during the treatment.

Additionally, by setting or adjusting beam impingement angle of the beams emitted by the output cables 1501a, 1501b, a depth of tissue treatment can be controlled. In particular, by decreasing the angle of the multiple beams relative to vertical, the overlap region can be formed deeper into the tissue and/or extend deeper into the tissue. Advantageously, by overlapping the beams deeper in the tissue T, more tissue T can be treated during a procedure. Additionally, deeper treatment areas can target different, deeper tissues T than single beam systems or systems having a shallow overlap region. Thus, particular selection or adjustment of slant incident angles, including, for example, from about three (3) degrees to about 75 degrees, can provide high EMR power targeted at a desired depth in the desired tissue T without overheating the impingement surface S.

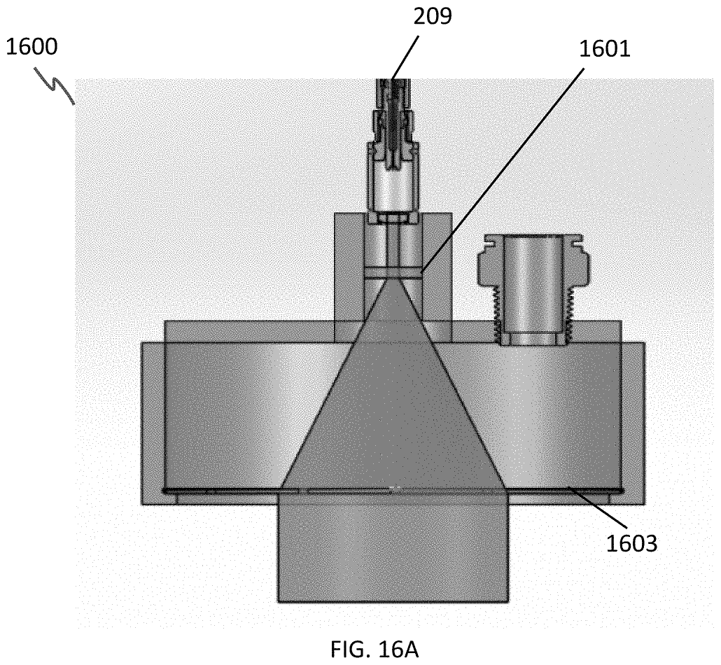

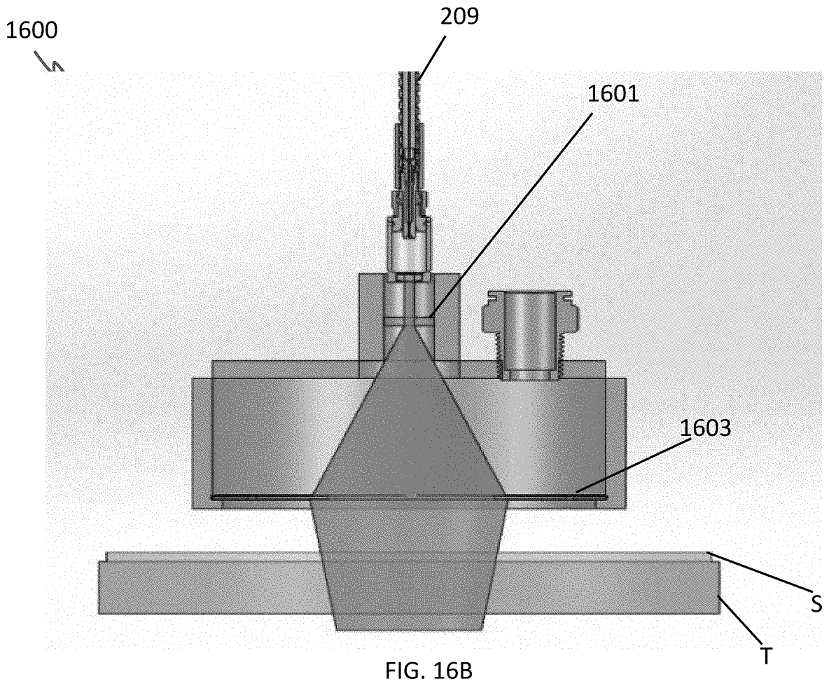

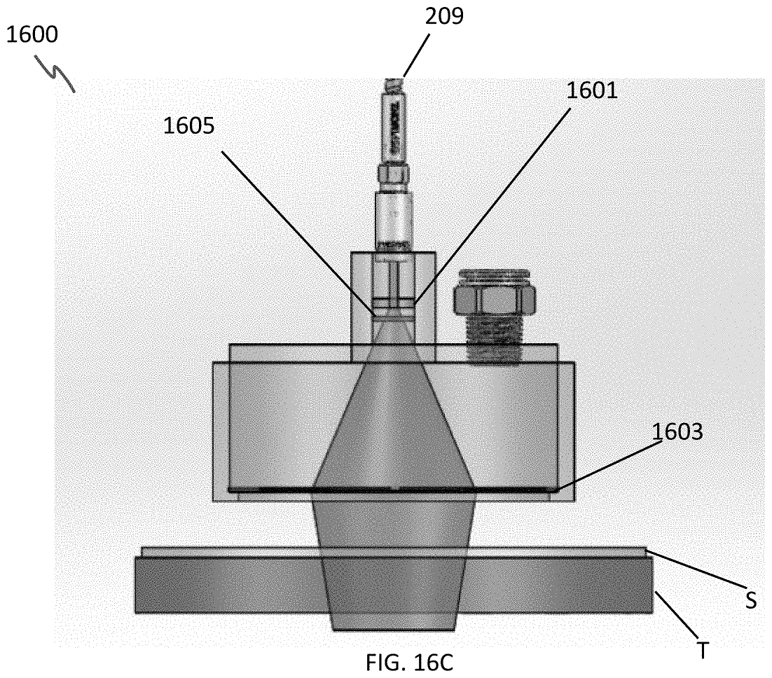

Referring now to FIG. 16A, in some embodiments, a device 1600 can include one or more optical elements for expanding, homogenizing, and refocusing EMR energy to aid treatment. In particular, a small, straight beam directed at a surface S to be illuminated can concentrate the EMR power in a small treatment area, making temperature management difficult and requiring additional movement and time to treat a target tissue T. Thus, in some embodiments, the device 1600 can include a beam expander 1601 to expand a size of a beam emitted by the common output cable 209. In particular, the beam expander 1601 of FIG. 16 is shown as a diffractive optical element (DOE) beam expander 1601. However, it will be apparent in view of this disclosure that any beam homogenizer, beam expander, or combination thereof can be used in accordance with various embodiments.

For applications where the target tissue T is beneath a surface S to be illuminated (e.g., where apoptosis of adipose tissue is desired), a beam expander 1601 alone would cause the beam power to be most diffuse in the target tissue T. Such a configuration makes heat management of the illuminated skin more difficult because the skin surface S is exposed to more concentrated beam power and thus heats up more quickly than the target tissue T. Therefore, in some embodiments, the device 1600 can also include a Fresnel objective lens 1603 for refocusing the expanded beam. As shown in FIG. 16B, in some embodiments, adjusting a spacing between the DOE beam expander 1601 and the Fresnel objective lens 1603 can adjust the focus. Thus, in some embodiments, the beam can be adjusted to be narrower (more concentrated) in the target tissue T and more diffuse at the surface S such that the skin surface S heats more slowly than the target tissue T. Referring now to FIG. 16C, in some embodiments, a negative Fresnel lens 1605 can be positioned between the beam expander 1601 and the Fresnel lens 1603 to permit additional beam shaping.

Referring again to FIG. 1, the system 10, in some embodiments, can include one or more sensors 1000 for monitoring operational conditions such as temperature of the treatment area. In some embodiments, the sensors 1000 can be configured to provide real time feedback to the computing device 107 so that the computing device 107 can, if desired, provide instructions to one or more components of the system 10 to alter one or more operational properties of the system 10 in response to the feedback. For example, in some embodiments, the positioning apparatus 900 can be instructed to scan the target area faster or slower to decrease or increase dwell time, move the device 950 closer to or further away from the target surface, reposition the device 950, temporarily suspend treatment, terminate treatment, or increase or decrease cooling flow through a patient cooling system.

To the extent that patient temperature data is required, in some embodiments, to maintain a therapeutically acceptable temperature range, a subcutaneous temperature prediction sensor 1000 can be provided. Some rely on blackbody radiation signals in the microwave region. Others employ temperature sensors, in combination with estimated skin and tissue thermal conductivity, to predict the core temperature. Some devices have attached heated sensors to the skin with temperature sensors to predict core temperatures. Other approaches have monitored the skin surface temperature and the energy input.

Invasive temperature measurements are possible but not preferred due to the associated risks, and desire for a fully non-invasive hyperthermia treatment. Elaborate instruments such as MRI (Magnetic Resonance Imaging) or advance ultrasonic devices are capable of these measurements but involve expensive and large devices which are also not readily used during many treatments.

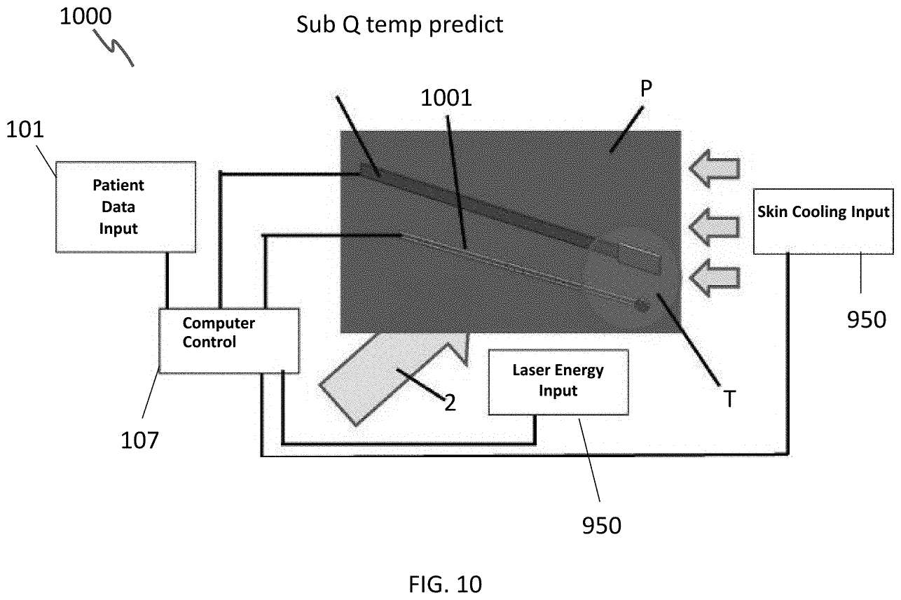

Referring to FIG. 10, in some embodiments, a non-invasive sensor 1000 for measuring a core body fat temperature of a patient can be used. The sensor 1000 can include a temperature sensor 1001 for measuring skin surface temperature and a heat flux sensor 1003 for measuring heat flow into or out of the treatment site. In some embodiments, the temperature sensor 1001 can include, for example, a thermocouple or a non-contact pyrometer. In some embodiments, the heat flux sensor 1003 can include, for example, a thermopile or a Seebeck effect sensor.

The sensor 1000 can then continuously monitor temperature and heat flux of the patient during treatment and feed that data back to the computing device 107 for processing. The temperature and heat flux data can be synthesized in an algorithm with user input data such as patient skin type, age, size, body fat percentage, etc. to estimate a temperature of the target subcutaneous fat. The computer system 107 can then adjust one or more operating parameters such as pulse length, EMR source activation, EMR source power, treatment duration, cooling airflow, scanning speed of the positioning apparatus, etc. to manage the temperature in response to the sensor 1000 feedback. Although shown as including both a temperature sensor 1001 and a heat flux sensor 1003, it will be apparent in view of this disclosure that, in some embodiments, the sensors 1000 may include only a temperature sensor 1001 or only a heat flux sensor 1003.

In some embodiments, the continuous temperature monitoring can begin with a numerical finite element simulation of fat region heating under EMR illumination to predict temperature over time and EMR source modulation. In particular, EMR source heating is applied in time dependent modulation and diminishes with depth of penetration. As the procedure progresses, skin temperature and skin heat flux are measured for the patient using the temperature sensor 1001 and the heat flux sensor 1003. Then, the temperature and heat flux data, the patient's unique data, and the finite element model are entered and combined in an overall algorithm to control the radiation input actively and maintain fat temperature in the effective range.

The measured parameters of a patient's skin temperature and skin heat flux in cooled regions can be measured several ways. Skin surface temperature can be made by a non-contact optical pyrometer recording in the radiated region, or a thermistor or thermocouple package. Temperature will be monitored before, during, and after EMR source irradiation. The rate of change of the skin temperature is monitored in the algorithm. The skin heat flux is derived in a non-contact method using the surface temperature measurement in combination with actively monitored cooling flow rate. When the two measurements are included in a heat transfer algorithm, calculation of skin heat flux is possible. Alternatively, a surface heat flux sensor can provide heat flux data.

Patient data used in this algorithm includes skin type and pigment, gender, age, size, weight, body mass index, and possible pretreatment history and skin distinctions. When available, more detailed tissue data can be entered. Tissue profiling collected from MRI's or ultrasonic devices can also provide accurate parameters to be incorporated into the tissue model. Other technologies such as non-invasive body core temperature measurement instruments that use black body radiation in the microwave region can be applied. Patient factors such as skin pigment characterization are important to estimate the anticipated EMR transmission and absorption values.

The algorithm is used to control the EMR energy delivered to a treatment area, known as fluence, in watts per square centimeter, as well as the exposure durations. The hyperthermia adipose reduction in some embodiments is done with on-off modulations and possible movement of beam location, which returns to reheat a region to maintain effective temperature range. The skin cooling is expected to be controlled based on skin surface temperature feedback for comfort level (e.g., 30° C.) and maximum safe temperature (e.g., 43° C.). The entire treatment period can last from several minutes to more than 30 minutes.

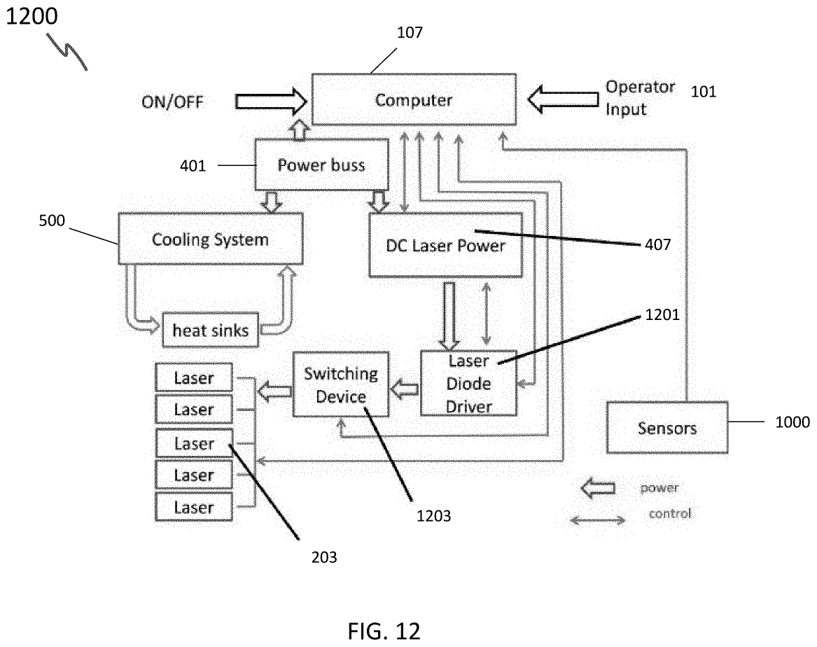

Referring now to FIG. 12, a schematic of a system 1200 for electronics and control of a multifunction aesthetic system having a single diode driver is provided. In particular, ADC 411 (analog to digital converter) can operate one or more laser sources 203 from a shared diode driver module. In this case, the one or more laser sources 203 have the same voltage/current requirements and are operated from a single diode driver. In some embodiments, the system 1200 is substantially similar to the system 400 of FIG. 4. However, the system 1200 of FIG. 12, includes a single diode driver 1201 and a switching device 1203 interposed between the diode driver 1201 and the laser sources 203 to permit the diode driver 1201 to selectively drive a desired one of the laser sources 203 (in the event that there is more than one).

The diode driver 1201, in some embodiments, can be substantially similar to the diode drivers 409 discussed above in connection with FIG. 4. The switching device 1203, in some embodiments, can be configured to switch the driver 1201 between the diode load of each laser source 203 if/as required. In some embodiments, the switching device 1201 can include one or more high current mechanical relays, one or more solid state relays (SSR), or both.

The switching device 1203 can be placed on ‘high side’ of the diode driver and the relays can be selected one at a time to drive a particular laser source 203. The relays must be capable of handling the current driven to the selected laser source 203. The relays or SSRs can be used as a safety interlock (emergency power cut) for the laser sources 203 as well. However, in the configuration of FIG. 12, multiple laser sources 203 cannot be driven by selecting more than one relay at a time. Such a configuration would place the laser sources 203 in parallel with each other and the driver 1201. Even if the driver 1201 is capable of sufficient current, there is no passive or active load sharing between the two laser sources 203. Because one of the diodes will have a lower resistance, that device will ‘hog’ the current, over power, and burn out, leaving the second channel to do the same. Because such burnout can happen very quickly (seconds), the switching device 1203 must be configured to select only one diode at a time. Additionally, switching the diode channel must occur when the driver is off. In particular, diode laser sources 203 operate at a near short (about 3 milliohms for a diode bar). Therefore, if the output of an active driver is switched from an open load to a diode load, a large overcurrent spike will occur, likely damaging or destroying the diode.

When deciding between SSR and mechanical relays, SSRs tend to be faster, more reliable, and don't typically require electrically isolated control lines. However, isolated input SSRs allow the use of a single driver for several diodes with less concern for ground loop issues. In addition, in the event of a failure, an isolated SSR input will provide a buffer for the sensitive control circuitry.

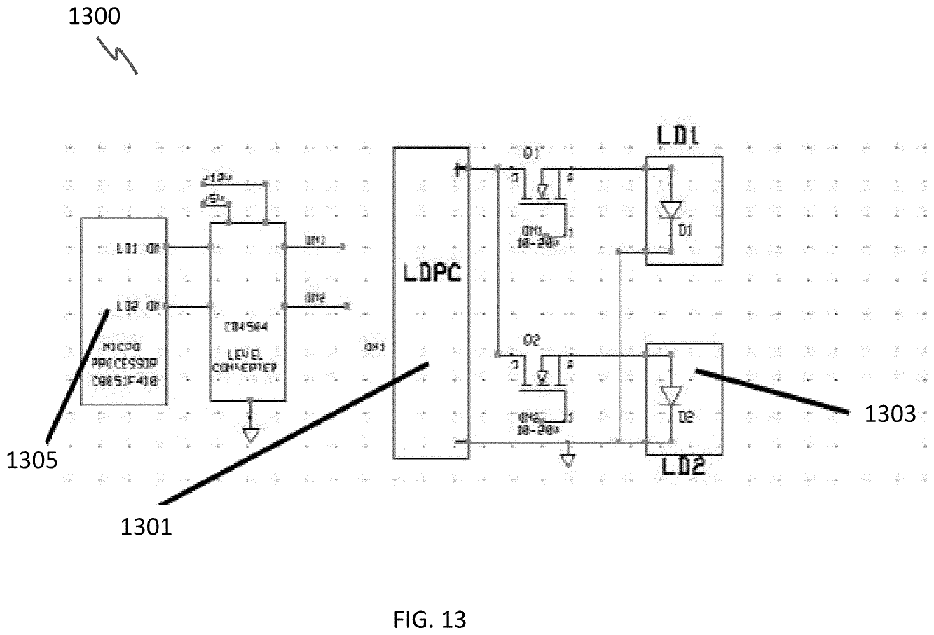

Referring now to FIG. 13, in some embodiments, the switching device can employ a single Diode Driver Printed Circuit (DPC) 1301 to power one or more EMR sources 1303 is shown. The high current capacity FET's can be used as switching devices to activate and power the selected EMR source. For example, the FETs can be Enfineon EPT004N03₁ rated at 30V and 320 A, resistance is 0.0004 ohms. At 70 A the FETs can drop about 30 mV and dissipate about 2 W. The FETs can also be run with a 12V control signal as shown. Although the diagram in FIG. 13 shows only two drivers (LD1 and LD2), but the same concept can be applied to drive multiple EMR sources. The control input to the switching FET's is routed from the processor 1305. This design approach eliminates the need for switching relays with the command signal driving only the selected driver and therefore activating that EMR source.

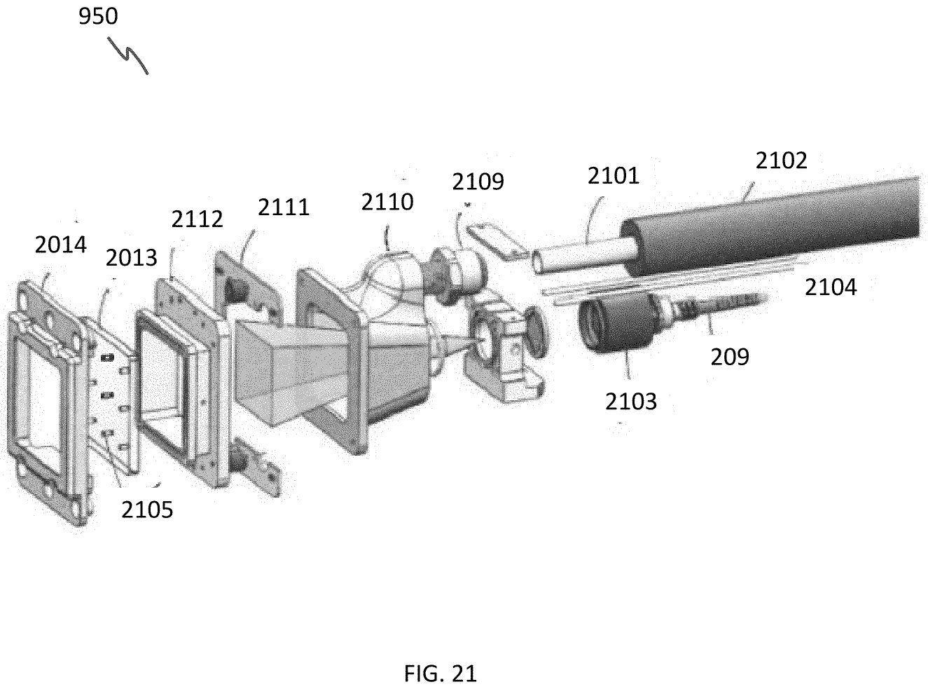

Device 950, sometimes referred to as the treatment head, as used in some embodiments, is shown in FIG. 21 in an exploded view. In some embodiments, cooled air supply 2101 can be received from an air chiller. The air supply 2101 is preferably surrounded by insulation 2102. In some embodiments, output cable 209 can deliver EMR from the at least one EMR source 203. The output cable 209 can contain one, two, or more fiber optics and can be connected to device 950 via fiber optic connector 2103. The positioning apparatus 900 (or arm housing), not shown, can be connected to device 950 to assist in facilitating movement of the device 950. In some embodiments, air supply 2101 can be contained in a tube or mesh for convenience (not shown) and include one or more rods, 2104, for stiffening the connection between the positioning apparatus 900 and the device 950. Rods, 2104, can be fabricated from fiberglass, metal such as steel, stainless steel, nitinol, or the like, polymers or reinforced polymers. Air supply 2101 can be connected to plenum 2110 via connection 2109. As will be shown and discussed later, the EMR from output cable 209 goes first through a lens that collimates the beam to a cylinder. The EMR then goes a diffuser which produces, in some embodiments, a diverging square beam. In some embodiments, circuit board 2111, which contains one or more sensors (not shown) can be placed against plenum 2110 and held in place by plate 2112. Window 2113 can be placed in the opening of plate 2112 and held in place by base 2114. Seals can be employed to make the plenum assembly air-tight. These seals can be O-rings or other similar materials between the window 2113 and the plate 2112 and the plate 2112 to the plenum 2110. In order to create the air-cooling impingement flow, window 2113 have one or more openings 2105 that allow for air flow. Preferably, other than openings 2105, the plenum and system can be air tight. In some embodiments, diffuser 2202 and window 2113 (along with the plenum 2110 body) can form the air tight space for the chilled air.

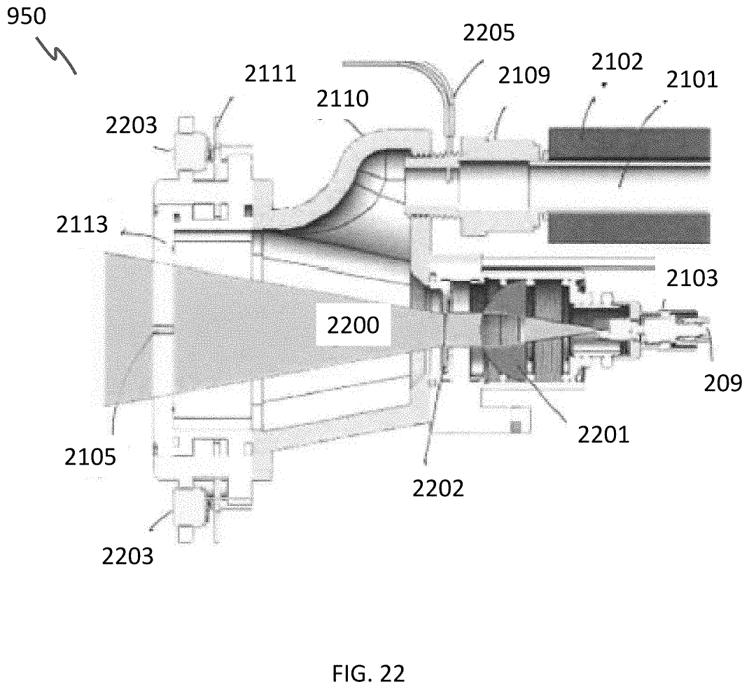

Referring to FIG. 22, a cross section of some of the components of device 950 from FIG. 21 is depicted. As in FIG. 21, chilled air supply, 2101 is surrounded by insulation 2102 and connected to plenum 2110 via connection 2109. In some embodiments, a temperature sensor 2205 can be located within the chilled air path to measure the air temperature of chilled air 2101. Temperature sensor 2205 can be any combination of sensors, for example, a thermocouple or similar instrument. The output from temperature sensor 2205 can be fed back to computing device 107 (FIG. 1) for processing. Output cable 209 is connected via connector 2103. In some embodiments, the EMR beam 2200, which is emitted from the fiber core of connection 2103, can expand as it moves further away from the end of the fiber core. Lens 2201 can be used to modify the expanding cylindrical beam to a columnar beam. The lens 2201 can be any combination of lens types, for example, lens 2201 can be a Fresnel Lens.

In some embodiments, diffuser 2202 can be used to convert the columnar EMR beam from a cylinder to a square beam. The resulting beam can be square, a diverging square, a converging square, a rectangle, a diverging rectangle, or a converging rectangle. In some embodiments, refractive diffuser optical element or an etched micro lenses and prisms can be used to create the beam pattern. In some embodiments, diffuser 2202 can be an engineered diffuser which employs refraction with micro arrays of lenses and prisms to produce the desired EMR beam shape. The diffuser 2202 can convert the beam into a uniform beam that provides a uniform treatment within the cross-section side dimension of the beam. These engineered diffusers are wavelength independent, have a high efficiency, and can produce a ‘top hat’ beam with uniform power density. A ‘top hat’ beam is an EMR beam has a near-uniform fluence (energy density) across the entire beam. In some embodiments, the resulting beam is a 20° diverging square beam, meaning that all four sides of the beam increase at an angle of 10°. In further embodiments, the beam measures 4.3 cm×4.3 cm when it is emitted from device 950.

In some embodiments, the device 950 can include a blocking filter (not depicted) to filter out light that is reflected from sources that are not meant for interpretation. For example, the blocking filter can be used to increase the accuracy of a proximity sensor which relies on time of flight measurement to establish a distance between the device 950 and a surface of the skin. In this example, the blocking filter will filter which light reaches the proximity sensor. In some embodiments, the blocking filter can also protect one or more sensors against dust. The device 950 can also include a plurality of other sensors or a sensor array having a plurality of sensors. For example, the device 950 can include a sensor array having at least one of a skin temperature sensor, an air-cooling temperature sensor, air flow sensor, laser power sensor, a location sensor, and a proximity sensor. Also shown in circuit board 2111 can have two skin temperature sensors, 2203. After EMR beam passes through diffuser 2202, it passes through window 2113 having one or more openings 2105.

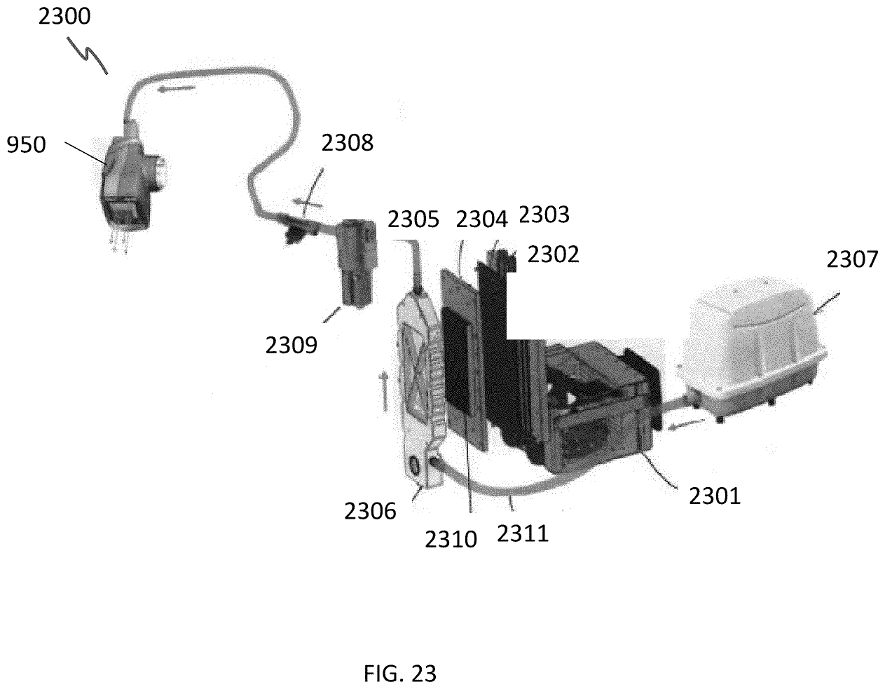

Referring to FIG. 23, in some embodiments, an air chiller system 2300 can be used to cool aspects of the device 950 and/or a treatment area targeted by the device 950. The air chiller system 2300 can be similar to the chillers discussed in the embodiments shown in FIGS. 5 and 7 with the exception that the cooling fluid is not circulated to the devices to be chilled. Rather a cold plate heat exchanger is used to provide cooling. Refrigeration unit 2301 can include a compressor, a condenser, an evaporator and fan unit. In some embodiments, to improve air quality, the fan can include a filter to capture particles, bacteria, and viruses, thereby preventing circulation of such particles, bacteria, and viruses through air surrounding the system. The refrigerant can be circulated to evaporator 2302 in tubes 2303. This provides the cooling for the system—the air system is shown in FIG. 23. Thermal pad 2304 touches evaporator 2302. Thermal pad 2304 is optional; it is shown here as it improves thermal conduction. In some embodiments, heat exchanger 2305 can be against thermal pad 2304. An open side of air plenum 2306 is sealed with heat exchanger 2305. The side of heat exchanger 2305 that is within plenum 2306 optionally contains fins 2310 or other structures to promote heat exchange. Air pump 2307 pumps air through hose 2311 to air plenum 2306 where it is cooled. The chilled air exits air plenum 2306 via tube 2312 to water trap 2309 where liquid water is removed. It then flows to flow sensor 2308. This flow signal may be sent to computing device 107 (e.g., as shown in FIG. 1) and then flow to device 950.

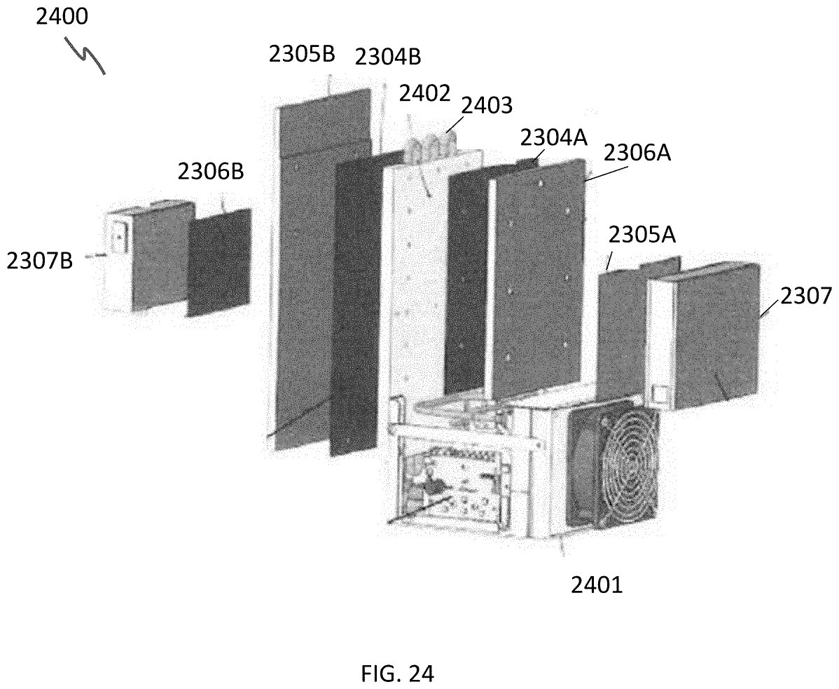

Referring to FIG. 24, in some embodiments, a laser chilling system 2400 can be used to cool aspects of the device 950 and/or a treatment area targeted by the device 950. A refrigeration unit 2401, as has been discussed herein, can be used to provide refrigeration to the laser chilling system 2400. Cooled refrigerant can be circulated via tube 2403 to evaporator 2402. In some embodiments, as shown in FIG. 24, dual cooling systems can be used for two or more laser units 203 (shown here are 2307A and 2307B). While two systems are shown, a single system is also contemplated. While only one laser unit 203 is shown on each of thermal pad 2306A and 2306B, more than one laser unit 203 could be mounted on either one or both. In some embodiments, adjacent to evaporator 2402, can be thermal pads 2304A and 2304B and then cold plates 2305A and 2305B, respectively. Laser 2307A has thermal pad 2306A which is held against cold plate 2305A. Similarly, laser 2307B is against thermal pad 2306B which is against cold plate 2306B. Thermal pads 2306A and/or 2306B are optional; they are shown here as they improve thermal conduction.

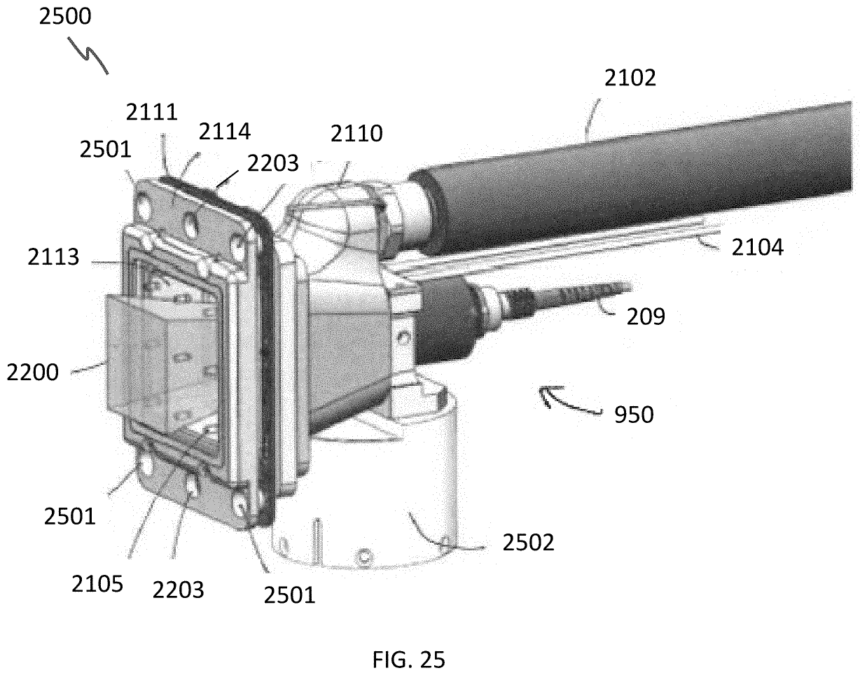

Referring to FIG. 25, and example depiction of device 950 is shown. In some embodiments, the air supply inside insulation 2102 and the EMR in output cable 209 can be delivered to device 950. A protective sheath (not shown) can surround the air supply along with rods 2104 which provide strain relief. The chilled air is supplied to plenum 2110 which is air tight except for openings 2105 in window 2113. Circuit board 2111 is shown with two skin temperature sensors 2203 and four proximity sensors 2501. While six sensors are shown, any number and any combination of temperature and proximity sensors can be used. The proximity sensors can include any combination of sensors capable of determining a distance between the treatment head of the device 950 and a treatment surface. For example, the proximity sensors can include time of light diodes, echolocation, ultrasound, etc. In some embodiments, the proximity sensors can be used to maintain a consistent height of separation between the device 950 and a treatment surface by providing feedback to a controller which can adjust a height of the device 950 to maintain said consistent height. In some embodiments, a plurality of skin temperature sensors can be positioned around a perimeter of the treatment head. Sensor output is fed to computing device 107. Openings in base 2114 are provide for sensors 2203 and 2501. Laser beam 2200 is delivered through window 2113 to the treatment area.

Connector 2502 can be used to connect device 950 to positioning apparatus 900. In some embodiments, beam 2200 can be centered in the treatment head. In some embodiments, the beam 2200 can be coaxial with the chilled air flow so that the part of the treatment zone that is receiving the beam 2200 is cooled. As discussed herein, chilled air flow can be provided to cool any combination of the device 950 and the skin of a patient, however, chilled airflow is not intended to be limited to any temperature of air. For example, the chilled airflow is not limited to air that is refrigerated but can include any combination of airflows, air velocities, air volumes, and air temperatures, that can provide cooling. In some embodiments, the device 950 can include one or more air cooling temperature sensor proximal to the airflow exiting the device 950. Other types of air sensors can also be used to monitor the airflow from the device 950. For example, the device 950 can include any combination of air flow sensor, air flow rate sensor, air velocity sensor, etc.

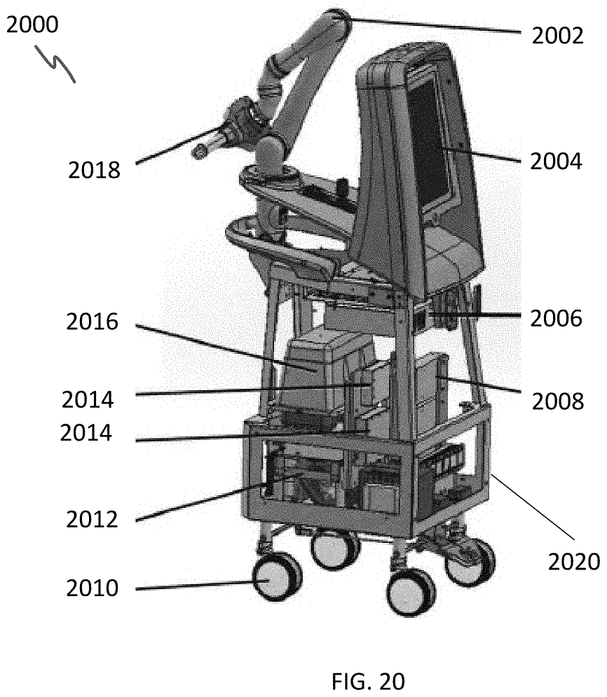

Referring to FIG. 20, an example embodiment of a treatment system 2000 is depicted. The system 2000 can include the various components of the treatment systems discussed herein. Some of the components include a DC power supply 2008 which, in some embodiments, converts AC power supply to DC to power the laser driver 2006 and the one or more lasers 2014. The system 2000 can also include an air pump 2016 which supplies air for cooling the treatment area when the system is in use. In some embodiments, the system 2000 includes a laser chiller 2012 to provide cooling to the one or more lasers 2014 and optionally to the laser driver 2006 and/or DC power supply 2008. The system 2000 can also include a ser interface 2004 that allows the user/physician to input data into the system and to control particular elements of the system 2000. In some embodiments, the system includes a movable robotic arm 2002 moves treatment head 2018 into proximity with the treatment area when the system 2000 is in use. The entire system 2000 may be made portable or movable through the use of castors or wheels 2010. In some embodiments, these components are mounted on or carried on frame 2020. While not shown, frame 2020 may be covered with a metal or plastic skin or covering to protect the internal components.

In some embodiments, in operation, the system 2000 can be designed to generate a square or rectangular EMR beam, which may be preferred to a round or rounded EMR beam. As a square or rectangular beam moves across the treatment surface, all of the treatment surface that is within the beam will receive approximately the same amount of energy as the beam has a constant length and width. If a round or rounded beam is used, the part of the treatment zone that lies in the axis or diameter of the beam (parallel to the direction of travel) will receive the maximum energy and the part of the treatment zone that lies at the edge of the EMR beam (at the ends of a diameter perpendicular to the direction of travel) will receive minimal energy.

In some embodiments, window 2113 can measure approximately 2.5 in×2.5 in and can have nine holes, each approximately 0.090 in in diameter spaced approximately 0.8 inches apart. The window 2113 can include any number of holes of any combination of dimensions to provide a high-volume airflow therethrough for cooling. For example, by supplying an air flow of 7 to 8 cubic feet per minute (CFM) (200 liters per minute (LPM) to device 950, the chilled air jet impingement output has a velocity of more than 60 meters per second. This configuration produces a cooling area of almost 3 in. by 3 in. to efficiently cool the treatment surface. As discussed herein, air is a useful cooling fluid as it does not interfere with the EMR delivery.

In some embodiments, device 950 also includes one or more indicators. The indicators can alert a user whenever EMR is being emitted. For example, the one or more indicators can be lasers or LEDs that light up when the EMR is being emitted. This alerts the patient and user/physician that the treatment is ongoing even if the EMR beam itself is not visible. In some embodiments, the one or more indicators can include an alignment light to assist a user with the alignment the aesthetic treatment device, for example, when registering or mapping one of the plurality of markings to initiate the registering of the treatment area.

In some embodiments, a computer control system or computing device (107 in FIG. 1) can control many aspects of the treatment systems discussed herein. For example, some combination of a user interface and user controls (e.g., joystick, buttons, switches, trackball, etc.) can be provided to control many aspects of the treatment system. The user interface 101 (FIG. 1) may be a touch screen. In some embodiments, when the system is started, the refrigeration units may start as the chilled air system needs to be operational in order to cool the treatment surface, such as the skin, when the EMR source is activated. Upon starting, the user/physician may be asked to input certain data, for example, patient data such as height, weight, skin type, age, body contour map, body location sensing data, etc. as well as procedural parameters such as desired beam power, procedure type, wavelength or wavelengths to be applied, pulse duration, treatment duration, beam pattern, treatment area temperature, therapy parameters, skin temperature data (generic or patient specific), skin temperature heat flux data (generic or patient specific), timing data, etc. In some embodiments, data such as: male or female; and treatment option such as fat reduction or wrinkle reduction is needed by the system and other aspects are preprogrammed into the control system.

In some embodiments, the user/physician can input the treatment area into the control system. This can be accomplished by moving the treatment head to one corner of the treatment area and giving an indication to the control system by pressing a button or box on the user interface. In some embodiments, the device 950 is manually moved by the user/physician having a joystick in communication with the control system or by using arrow buttons or the like on the touch screen. The user/physician then indicates the other corners of the treatment area to the control system in the same fashion. While the treatment area may be any shape, at least three corners must be indicated. At this point, the user/physician may activate the system by pressing a start button or box on the user interface. As the treatment uses EMR such as laser, the system may require the user/physician acknowledge that everyone in the treatment room has proper eye protection. In some embodiments, the user/physician can use a template as discussed in greater detail herein to assist in marking the treatment area.

Once started, the control system can generate one or more treatment zones in the treatment area. The control system can move the robotic arm and device 950 to a position over the first treatment zone, and in some embodiments, start the cooling air flow and the EMR. While in operation, the proximity sensors can send information to the control unit to ensure that the face of device 950 stays within the proper distance away from the treatment surface. In some embodiments, the face of device is kept approximately 0.7+/−0.25 inches from the treatment surface. This distance is chosen to ensure that the air-cooling system works properly and the EMR beam is of the proper size and to ensure that the size of the beam will track within the treatment are according to a designated pattern (e.g., as shown in FIG. 18). With an air impingement system, the distance from the holes in the face of device 950 to the skin affects the rate of cooling. In some embodiments where a diverging EMR beam is used, being too close to the surface will result in an over-concentration of energy and being too far away from the treatment surface will result in the beam being too spread out which could cause the beam to have too little energy to provide the selected treatment or could cause parts of the beam to be outside of the cooling area.

While the EMR beam is active, a number of safety systems may be active. As discussed above, the proximity sensors maintain the device 950 a proper distance away from the treatment surface. Temperature sensors measure the surface temperature of the treatment area to ensure that the surface does not get above a specified temperature. The control system may use the surface temperature to calculate a subcutaneous treatment zone temperature. In some embodiments, an air temperature sensor may measure the temperature of the chilled air to ensure that it is within a proper range. The EMR system may have a sensor to detect the power of the EMR to ensure that it is within a specified range. There may be temperature sensors on the EMR generators and/or power supplies to ensure that they do not overheat. If any of the measured values are outside of a specified zone, the control system may notify the physician/user, may stop the EMR delivery, may change operational aspects of the system to correct the deviation, or may cease operation of the device. For example, in some embodiments, if the surface temperature of the treatment area gets too high, they system may turn the laser off, may move the laser to another treatment zone, may increase the rate of cooling, and/or may reduce the power of the EMR that is being delivered. In some embodiments, the application of energy to the treatment zone can be restarted when the temperature of the treatment zone surface is lower than the maximum surface temperature.

In some embodiments, the treatment zone can be a rectangle that has a length that is an approximate multiple of the length of the EMR beam and a width that is an approximate multiple of the width of the EMR beam. The multiple can be a whole or other number. In some embodiments, the treatment zone is four times the length and two times the width of the EMR beam. After starting the system, the device 950 is moved to the first treatment zone and the treatment begins. The control system moves the EMR beam in a predetermined pattern and at a predetermined rate in the treatment zone. In some embodiments, one complete scan (path over the entire treatment zone) is about 5 seconds or about 10 seconds. Once the treatment time is complete, the control system moves device 950 to the next treatment zone or, if the entire treatment area has been treated, the EMR is shut off and the device 950 is returned to the home position. The treatment time can be set as a fixed number of minutes, can be set as a number of minutes during which the treatment area or subcutaneous treatment zone is within the specified treatment temperature, or as a set number of scans over the treatment zone. In some embodiments, to speed up the overall treatment time, the control system may move device 950 to a subsequent treatment zone if the first treatment zone get too hot (rather than just shutting off the EMR).

In some embodiments, device 950 can have a visible laser, a LED or other indicator that provides a light source that acts as an aligning beam. When the device 950 is active, the aligning beam can generate a pattern that matches the treatment zone. In some embodiments, the generated pattern can be generated such that the beam will cover an entire area of the treatment area as it moved without having any overlap in previously treated areas, for example, as shown by the pattern in FIG. 18. The alignment beam can also show the user where the treatment is taking place. The aligning beam may also be used, when the EMR beam is not on, to assist the user in setting the treatment area. In some embodiments, the aligning beam emits red or green light. In some embodiments another alignment beam can include any combination of a visible laser, LED or other light source can be projected to show the treatment area or pattern.

Referring to FIGS. 26A, 26B, and 26C, example templates 2610, 2620, or 2630 that are used to indicate the treatment area are depicted. To indicate a treatment area, the appropriated template 2610, 2620, or 2630, can be placed on a patient's skin. The templates 2610, 2620, 2630 or other template designs may be made to be either single or multiple use and can be made of paper, plastic or metal. The templates 2610, 2620, 2630 can have some number of corner holes, for example, corner holes 2611, 2621, or 2631, and/or center holes 2612, 2622, or 2632. The corner and center holes can be provided to enable the user to make markings on the patient's skin to assist in mapping the treatment area, as discussed in great detail herein. For example, one of the templates 2610, 2620, 2630 can placed over the treatment site and two or more of the holes can be marked on the patient's skin. The markings on the patient can be used by the device to map the treatment area. For example, referring to FIG. 26C, the treatment area can set by moving the treatment head to a first corner 2631 of the treatment area and registering the first corner 2631, moving the treatment head to a second corner 2631 of the treatment area and registering the second corner 2631, moving the treatment head to a third corner 2631 of the treatment area and registering the third corner 2631, and moving the treatment head to a fourth corner 2631 of the treatment area and registering the fourth corner 2631. The four registered corners can then be used to generate the boundaries or perimeter for the treatment area or treatment zone.

In operation, to perform a treatment, a patient, positioned on a treatment platform, is placed near the treatment system as described herein. When a system, for example, system 2000 in FIG. 20, the system can be rolled to a position adjacent the patient. The treatment area is marked as described above. The user/physician activates the system through the user interface and the treatment head is moved to a position adjacent the treatment area. The treatment head may contain a visible light, for example the aligning light, either in the shape of an outline of or of the entire the treatment area or in the shape of a single point. If the treatment head light is in the shape of the outline of or the entire area of the treatment area, the treatment head is positioned such that the corners of the outline-shaped light match the markings on the skin and the user selects or saves the location through the user interface so that the system knows the treatment area. If the treatment head light is in the shape of a point, the user places the point of light onto two or more of the corner and/or center markings on the patient's skin and, through the user interface, saves or indicates the points so that the system's computer can determine the treatment area. Once the treatment area is marked, the user can begin the treatment.

The templates can be placed in different orientations for different procedures and for different patients. For example, for some patients who are being treated for belly fat, the template can be placed in a direction perpendicular to the longitudinal axis of the patient. For some patients, such as some women, the end of the template furthest away from the belly button will be 10° to 20° below the perpendicular axis. While some of the embodiments herein have been described for treatment of belly fat, the apparatus and systems described herein can be used for flank, leg, arm, back, or other fat.

Referring to FIG. 26A, a two by four treatment area template 2610 is shown. In this embodiment, the treatment beam is a rectangular or square shape. The length of the treatment area is equal to four times the length of the treatment beam and the width of the treatment area is equal to two times the width of the treatment beam, when the treatment beam is the appropriate distance from the treatment surface. Referring to FIG. 26B, a three by two treatment area template 2620 is shown and referring to FIG. 26C, a two by two treatment area template 2630 is shown. In these three examples, the treatment beam can be square or nearly square. In some embodiments, the treatment beam can be rectangular. The beam could be shaped such that the length is equal to two, three, four, or some other whole or non-whole number multiple of the beam width. In some embodiments where the length of the treatment beam is two times the width of the treatment beam, a template such as shown in FIG. 26C could be used. Instead of the beam travelling around in the treatment area, the beam would simply move back and forth, from one position to a second position and then back to the first position. While two by two, three by two, and four by two treatment areas are shown, other sized treatment areas such as three by three, four by three, or five by two could also be used. In some embodiments, non-whole number multiples of the beam width and/or length can be used to size the treatment area.

While some embodiments show the use of a template, in other embodiments the physician may draw the boundaries of the treatment area onto the skin of the patient with or without the use of a template. For example, the physician can create a customized template by using a marking device (dark ink, ultraviolet reflective ink, etc.) or other indicator that the system can see, the treatment head 950 can include sensors that recognize the markings and thus register or mark the treatment area by following the drawn pattern. In some embodiments, the physician can also create a custom template without using any markings. For example, with the assistance of a visible alignment light, the physician can more the treatment head to a location and click a button or other register a location with the device. This can be repeated until sufficient points have been registered to create a treatment area, for example, there or more registered points.

In some embodiments described above, lowering the EMR beam power during a treatment can help maintain the temperature of the skin at an acceptable level while the temperature of the subcutaneous treatment area can be raised to a therapeutically acceptable temperature. In other embodiments, the temperature of the skin can be kept at an acceptable level by implementing a cool down cycle intermittently during the treatment. For example, in a fat reduction treatment, applicants have found that the EMR beam can be kept at a power level of 150 W for the entire treatment as long as there are a number of cooling cycles. In some embodiments, the cooling cycle can run for 10 seconds and can include running the cooling air flow in the absence of the EMR beam while the treatment head continues to scan or move through the treatment area. In an example embodiment, a fat reduction treatment can include (i) scanning the treatment area with an EMR power level of 150 W for six minutes; (ii) running a cool down cycle for 10 seconds; (iii) scanning the treatment area for one minute at an EMR power level of 150 W; and (iv) repeating steps (ii) and (iii) until the subcutaneous tissue has been in the therapeutic temperature range for the target time period. By using this process, is has been determined that the deep fat tissue is maintained at a higher temperature while the skin and epidermis are both maintained at a sufficiently low level. This increased the effectiveness of the procedure while maintaining or even reducing the treatment time.

### Example Embodiment

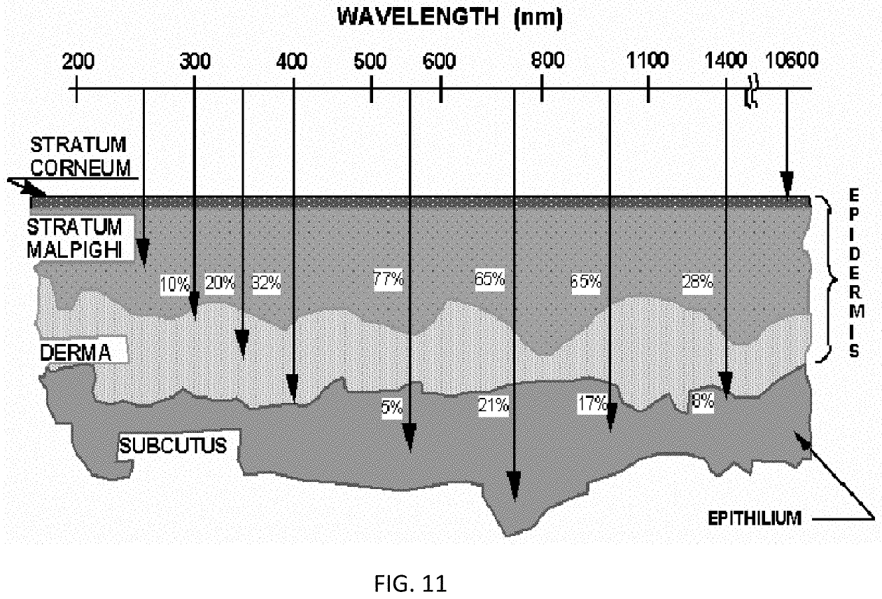

In one embodiment, it may be desirable to perform subcutaneous fat reduction and skin tightening simultaneously. However, as shown in the human tissue profile of FIG. 11, different EMR wavelengths have different expected penetration depths. In particular, FIG. 11 illustrates, by percentage, for each wavelength, the percentage of EMR energy penetrating to various depths. More generally the fat is typically more than 5 mm from the skin's surface. Thus, for example, a wavelength of about 1064 nm (e.g., 400 nm to 3000 nm or 900 nm to 1100 nm) can be selected for hyperthermia of fat tissue because it exhibits good transmission through the skin, epidermis, and dermis and deposits energy within the fat cells. On the other hand, skin tightening generally requires other wavelengths that exhibit higher absorption in the epidermis and dermis, where the collagen resides. Thus, for example, a wavelength of about 400 nm to about 3000 nm or about 1300 nm to about 1400 nm. These EMR beam wavelengths deposit more energy to the collagen, creating necrosis and eventually skin tightening from new collagen regrowth.

In such an embodiment, the controller 403 of the power and control electronics 400 of the multifunction aesthetic system 10 described herein can activate a first driver 409/laser source 203 pair to produce an EMR beam having a wavelength suitable for subcutaneous fat reduction while simultaneously activating a second driver 409/laser source 203 pair to produce an EMR beam having a wavelength suitable for skin tightening. In some embodiments, such a procedure can also be used in conjunction with other fat reduction techniques such as procedures using RF (radio frequency), MW (microwave), ultrasonic, or cryo (cold therapy) fat reduction methods.

In a further example, in some embodiments, the methods described above can be used to activate driver 409/laser source 203 pairs for emitting wavelengths suitable for performing any other procedure or combination of procedures including, for example, but not limited to, fat reduction, body skin tightening, facial skin tightening, skin resurfacing, skin remodeling, vein reduction or removal, facial pigment removal or reduction, hair removal, acne treatment, scar reduction and removal, psoriasis treatment, stretch mark removal, nail fungus treatment, leukoderma treatment, tattoo removal, or combinations thereof as discussed above.

In an example operation, a device 10 as shown in FIG. 1 can be used. A physician or user inputs information about a patient into user interface 101. In some embodiments, data such as: male or female; and treatment option such as fat reduction or wrinkle reduction is needed by the system and other aspects are preprogrammed into the control system. The patient is placed in a position adjacent to system 10 and computing device is activated. The user/physician moves positioning apparatus 900 and device 950 into position over the treatment area and sets the treatment area. Device 950 scans the desired treatment area and sends the data back to device 107. For example, the device 950 can the following information back to the system: skin temperature, head location, head height, whether the treatment laser is on or off, whether or not the cooling air is flowing, and cooling air temperature. The skin temperature can be used to determine if the treatment laser should be shut off if a skin temperature max setting has been reached. Head location and head height can be used by the head control system to ensure that the treatment is within the ‘template’ are and that the height of the head is proper. The cooling air flow and temperature can be safety measurements as the system will shut down if the flow is too low or if the air temperature gets too high (in both cases the air-cooling system will not be working as it should be).

Once the physician/user is certain that all parameters are set, the treatment begins. During treatment, proximity sensors 2501 on device 950 send data to device 107 so that positioning apparatus 900 keeps device 950 the proper distance from the treatment area. For some procedures, this will be between 0.5 and 1.0 inch, preferably 0.75 inches.

For treatments such as fat reduction or skin tightening, device 107 divides the treatment area into smaller treatment regions like shown in FIG. 18. In some embodiments, the beam is of a size and shape such that the treatment region has a length of four times the beam length (in some embodiments a square beam is used so that beam width equals beam length) and a width of two times the beam width. The laser beam is positioned in one corner of the treatment zone and the laser is activated. In some embodiments, the temperature sensors are positioned in the direction of beam travel so that one senses the skim temperature right in front of the beam and one measures the temperature right behind the beam. The beam can move at a rate such that the skin stays at a comfortable temperature—one that does not exceed 40° C.-43° C. — and the treatment area stays within the treatment temperature. For fat reduction therapy, it is desired that the subcutaneous fat tissue is heated to a temperature between 40° C. and 52° C. To keep the skin cool while the fat is being heated, chilled air at 5° C. is provide to device 950 under pressure and passes through opening 2105. The chilled air can be at a temperature more or less than 5° C. as long as the air flow keeps the skin at an acceptable temperature.

The pressure/volume of air and the distance of device 950 from the skin can be controlled so that impingement cooling is affected. As the air does not interfere with the EMR, multiple openings can be provided both within and outside the area of the EMR beam. As the beam moves through the path shown in FIG. 18, it will take more energy on the first scan than it will on subsequent scans as the fat retains the heat from prior scans. In some embodiments, the arm moves device 950 at a speed that doesn't allow the fat tissue to cool below 40° C. In some embodiments, device 107 steps down the power supplied to the laser after the first scan and down further after subsequent scans. In some embodiments, device 107 controls the distance of device 950 to the skin or the air flow rate, or the air temperature to control skin temperature. In some embodiments, the EMR beam can be shut off if the skin temperature gets above the preset maximum and will be turned back on once the temperature falls to an acceptable level. In some embodiments, when the first region has been treated (kept above a predetermined temperature) for the predetermined time, the device 107 will use positioning apparatus 900 to move device 950 to a second treatment region. This operation can continue until the entire treatment are has been treated.

In some embodiments, the systems and devices of the present disclosure can be used for causing thermal apoptosis in subcutaneous fatty tissues. The process can include moving a subcutaneous energy delivery device to a first treatment zone in a treatment area, applying energy to the first zone while moving the energy delivery device within the treatment zone at a rate that allows the subcutaneous tissue to reach a target temperature range, continuing the application of energy to the treatment zone while keeping the subcutaneous tissue within the target temperature range, the treatment zone having a treatment zone surface, and discontinuing the application of energy to any of the tissue in the first treatment zone that have been in the target temperature range for a target treatment time.

In some embodiments, less energy can be delivered on a scan as compared to a prior scan. The process can also include discontinuing the application of energy to any of the tissue in the first treatment zone when the temperature of the treatment zone surface. The target temperature range during the process can be 42° C.-51° C. and the energy can be applied to an area that is smaller than the area of the treatment zone. In some embodiments, the energy delivery device can complete a scan by applying energy to the entire area of the treatment zone. Multiple scans may be needed to raise the temperature of the subcutaneous tissue to the target temperature range. In some instances, less energy can be delivered by the energy delivery device after the subcutaneous tissue has reached the target temperature range than prior to the subcutaneous tissue has reached the target temperature range. The energy delivery device can also include a temperature sensor for sensing the temperature of the treatment zone surface. In some embodiments, the application of energy to the treatment zone can be stopped when the temperature of the treatment zone surface is higher than a maximum surface temperature. The application of energy to the treatment zone can be restarted when the temperature of the treatment zone surface is lower than the maximum surface temperature. The maximum surface temperature is 42° C. and the temperature of the subcutaneous tissue can be calculated from the temperature of the treatment surface temperature.

In some embodiments, the systems and devices of the present disclosure can be used for causing thermal apoptosis in subcutaneous fatty tissues. The process can include (i) moving a subcutaneous energy delivery device to a treatment area, (ii) applying energy and cooling air to the treatment area while moving the energy delivery device within the treatment area to raise the temperature of the subcutaneous fatty tissue to a therapeutically acceptable range, (iii) stopping the application of energy to the treatment area while maintaining the application of cooling air while moving the energy delivery device within the treatment area, and (iv) applying energy and cooling air to the treatment area while moving the energy delivery device within the treatment area. The temperature of the fatty tissue can be maintained in the therapeutically acceptable range during the treatment. The fatty tissue is maintained above 42° C. and the application of energy can be stopped for 5 to 15 seconds. The can also include repeating steps (iii) and (iv) until the fatty tissue has been maintained within the therapeutically acceptable range for a predetermined period of time.

While the present disclosure has been described with reference to certain embodiments thereof, it should be understood by those skilled in the art that various changes may be made and equivalents may be substituted without departing from the true spirit and scope of the disclosure. In addition, many modifications may be made to adapt to a particular situation, indication, material and composition of matter, process step or steps, without departing from the spirit and scope of the present disclosure. All such modifications are intended to be within the scope of the claims appended hereto.

As utilized herein, the terms “comprises” and “comprising” are intended to be construed as being inclusive, not exclusive. As utilized herein, the terms “exemplary,” “example,” and “illustrative,” are intended to mean “serving as an example, instance, or illustration” and should not be construed as indicating, or not indicating, a preferred or advantageous configuration relative to other configurations. As utilized herein, the terms “about,” “generally,” and “approximately” are intended to cover variations that may existing in the upper and lower limits of the ranges of subjective or objective values, such as variations in properties, parameters, sizes, and dimensions. In one non-limiting example, the terms “about,” “generally,” and “approximately” mean at, or plus 10 percent or less, or minus 10 percent or less. In one non-limiting example, the terms “about,” “generally,” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method for reducing subcutaneous adipose tissue in a patient, the method comprising:
providing at least one marking to define a treatment area within which adipose tissue is to be reduced;
directing electromagnetic radiation (EMR) from a housing to the treatment area to cause apoptosis of the adipose tissue, the housing being in spaced relation to the treatment area; and
permitting the housing to move within the treatment area while following a contour of the treatment area.

2. The method of claim 1, wherein the treatment area has a surface, and wherein following the contour of the treatment area comprises maintaining a predetermined distance between the housing and the surface.

3. The method of claim 2, wherein the predetermined distance is between 0.5 in and 1.0 in.

4. The method of claim 1, wherein the EMR has a wavelength of about 900 nm to about 1100 nm.

5. The method of claim 1, wherein defining the treatment area comprises registering the at least one marking by aligning the housing with the at least one marking to record its location.

6. The method of claim 1, wherein the step of permitting the housing to move within the treatment area comprises following a treatment pattern.

7. The method of claim 1, wherein the treatment area has a surface, and wherein the step of permitting further comprises:
determining a temperature of the surface; and
varying a scan rate of the housing based on the temperature of the surface.

8. A system for reducing subcutaneous adipose tissue in a patient, the system comprising:
an electromagnetic radiation (EMR) source configured to generate an EMR beam;
a housing configured to direct the EMR beam to a treatment area having subcutaneous adipose tissue; and
a positioning apparatus connected to the housing to guide the housing in spaced relation to the treatment area;
wherein the housing is configured to continuously direct the EMR beam to the treatment area to maintain the subcutaneous adipose tissue within a target temperature range.

9. The system of claim 8, wherein the positioning apparatus permits the housing to move in a predetermined pattern within the treatment area.

10. The system of claim 8, wherein the treatment area has a plurality of treatment zones, and wherein the positioning apparatus moves the housing between each of the plurality of treatment zones in a predetermined pattern.

11. The system of claim 10, wherein the positioning apparatus moves the housing over a given treatment zone at a scanning rate multiple times.

12. The system of claim 11, wherein a scanning rate of a subsequent pass over the given treatment zone is different than a scanning rate of a previous pass and wherein the scanning rate of the subsequent pass is faster than the scanning rate on the previous pass.

13. The system of claim 10, further comprising the EMR beam having a power level, and wherein the positioning apparatus moves the housing over a given treatment zone multiple times at different power levels.

14. The system of claim 8, further comprising the housing having a proximity sensor for determining a distance between the housing and a surface of the treatment area.

15. The system of claim 8, wherein the EMR beam has a wavelength of about 900 nm to about 1100 nm.

16. The system of claim 8, wherein the housing is connected to the EMR source with a fiber optic cable, and wherein the housing includes a lens for collimating the EMR beam and a diffractive or refractive diffuser for transforming the EMR beam into a rectangle shaped EMR beam.

17. A system for reducing subcutaneous adipose tissue in a patient, the system comprising:
a housing configured to direct an electromagnetic radiation (EMR) beam from an EMR source to a treatment area having adipose tissue, the housing having a proximity sensor for determining a distance between the housing and a surface of the treatment area;
a controller operatively connected to the housing, the proximity sensor, and the EMR source; and
a positioning apparatus connected to the housing and in communication with the controller so that the controller can guide movement of the housing within a defined boundary of the treatment area while maintaining a predetermined distance from the surface of the treatment area.

18. The system of claim 17, wherein the EMR beam has a wavelength of about 900 nm to about 1100 nm.

19. The system of claim 17, wherein the predetermined distance is between 0.5 in and 1.0 in.

20. The system of claim 17, wherein the housing is connected to the EMR source with a fiber optic cable, and wherein the housing includes a lens for collimating the EMR beam and a diffractive or refractive diffuser for transforming the EMR beam into a rectangle shaped EMR beam.
